SEQUENCE LISTING (11 sequences)

SEQ ID NO: 1 - DNA, 7996 bp, Homo sapiens
atatcccagtggcccccgtgcggcgactttagctgctgctgtctcagccgctccacagcgacggcggcggctgcggcttagtcggtggcggccggcggcggctgcgggctgagcggcgagtttccgatttaaagctgagctgcgaggaaaatggcggcgggaggatcaaaatacttgctggatggtggactcagagaccaataaaaataaactgcttgaacatcctttgactggttagccagttgctgatgtatattcaagatgagtggattaggagaaaacttggatccactggccagtgattcacgaaaacgcaaattgccatgtgatactccaggacaaggtcttacctgcagtggtgaaaaacggagacgggagcaggaaagtaaatatattgaagaattggctgagctgatatctgccaatcttagtgatattgacaatttcaatgtcaaaccagataaatgtgcgattttaaaggaaacagtaagacagatacgtcaaataaaagagcaaggaaaaactatttccaatgatgatgatgttcaaaaagccgatgtatcttctacagggcagggagttattgataaagactccttaggaccgcttttacttcaggcattggatggtttcctatttgtggtgaatcgagacggaaacattgtatttgtatcagaaaatgtcacacaatacctgcaatataagcaagaggacctggttaacacaagtgtttacaatatcttacatgaagaagacagaaaggattttcttaagaatttaccaaaatctacagttaatggagtttcctggacaaatgagacccaaagacaaaaaagccatacatttaattgccgtatgttgatgaaaacaccacatgatattctggaagacataaacgccagtcctgaaatgcgccagagatatgaaacaatgcagtgctttgccctgtctcagccacgagctatgatggaggaaggggaagatttgcaatcttgtatgatctgtgtggcacgccgcattactacaggagaaagaacatttccatcaaaccctgagagctttattaccagacatgatctttcaggaaaggttgtcaatatagatacaaattcactgagatcctccatgaggcctggctttgaagatataatccgaaggtgtattcagagattttttagtctaaatgatgggcagtcatggtcccagaaacgtcactatcaagaagcttatcttaatggccatgcagaaaccccagtatatcgattctcgttggctgatggaactatagtgactgcacagacaaaaagcaaactcttccgaaatcctgtaacaaatgatcgacatggctttgtctcaacccacttccttcagagagaacagaatggatatagaccaaacccaaatcctgttggacaagggattagaccacctatggctggatgcaacagttcggtaggcggcatgagtatgtcgccaaaccaaggcttacagatgccgagcagcagggcctatggcttggcagaccctagcaccacagggcagatgagtggagctaggtatgggggttccagtaacatagcttcattgacccctgggccaggcatgcaatcaccatcttcctaccagaacaacaactatgggctcaacatgagtagccccccacatgggagtcctggtcttgccccaaaccagcagaatatcatgatttctcctcgtaatcgtgggagtccaaagatagcctcacatcagttttctcctgttgcaggtgtgcactctcccatggcatcttctggcaatactgggaaccacagcttttccagcagctctctcagtgccctgcaagccatcagtgaaggtgtggggacttcccttttatctactctgtcatcaccaggccccaaattggataactctcccaatatgaatattacccaaccaagtaaagtaagcaatcaggattccaagagtcctctgggcttttattgcgaccaaaatccagtggagagttcaatgtgtcagtcaaatagcagagatcacctcagtgacaaagaaagtaaggagagcagtgttgagggggcagagaatcaaaggggtcctttggaaagcaaaggtcataaaaaattactgcagttacttacctgttcttctgatgaccggggtcattcctccttgaccaactcccccctagattcaagttgtaaagaatcttctgttagtgtcaccagcccctctggagtctcctcctctacatctggaggagtatcctctacatccaatatgcatgggtcactgttacaagagaagcaccggattttgcacaagttgctgcagaatgggaattcaccagctgaggtagccaagattactgcagaagccactgggaaagacaccagcagtataacttcttgtggggacggaaatgttgtcaagcaggagcagctaagtcctaagaagaaggagaataatgcacttcttagatacctgctggacagggatgatcctagtgatgcactctctaaagaactacagccccaagtggaaggagtggataataaaatgagtcagtgcaccagctccaccattcctagctcaagtcaagagaaagaccctaaaattaagacagagacaagtgaagagggatctggagacttggataatctagatgctattcttggtgatctgactagttctgacttttacaataattccatatcctcaaatggtagtcatctggggactaagcaacaggtgtttcaaggaactaattctctgggtttgaaaagttcacagtctgtgcagtctattcgtcctccatataaccgagcagtgtctctggatagccctgtttctgttggctcaagtcctccagtaaaaaatatcagtgctttccccatgttaccaaagcaacccatgttgggtgggaatccaagaatgatggatagtcaggaaaattatggctcaagtatgggtgggccaaaccgaaatgtgactgtgactcagactccttcctcaggagactggggcttaccaaactcaaaggccggcagaatggaacctatgaattcaaactccatgggaagaccaggaggagattataatacttctttacccagacctgcactgggtggctctattcccacattgcctcttcggtctaatagcataccaggtgcgagaccagtattgcaacagcagcagcagatgcttcaaatgaggcctggtgaaatccccatgggaatgggggctaatccctatggccaagcagcagcatctaaccaactgggttcctggcccgatggcatgttgtccatggaacaagtttctcatggcactcaaaataggcctcttcttaggaattccctggatgatcttgttgggccaccttccaacctggaaggccagagtgacgaaagagcattattggaccagctgcacactcttctcagcaacacagatgccacaggcctggaagaaattgacagagctttgggcattcctgaacttgtcaatcagggacaggcattagagcccaaacaggatgctttccaaggccaagaagcagcagtaatgatggatcagaaggcaggattatatggacagacatacccagcacaggggcctccaatgcaaggaggctttcatcttcagggacaatcaccatcttttaactctatgatgaatcagatgaaccagcaaggcaattttcctctccaaggaatgcacccacgagccaacatcatgagaccccggacaaacacccccaagcaacttagaatgcagcttcagcagaggctgcagggccagcagtttttgaatcagagccgacaggcacttgaattgaaaatggaaaaccctactgctggtggtgctgcggtgatgaggcctatgatgcagccccaggtgagctcccagcagggttttcttaatgctcaaatggtcgcccaacgcagcagagagctgctaagtcatcacttccgacaacagagggtggctatgatgatgcagcagcagcagcagcagcaacagcagcagcagcagcagcagcagcagcaacagcaacagcaacagcaacagcagcaacagcagcaaacccaggccttcagcccacctcctaatgtgactgcttcccccagcatggatgggcttttggcaggacccacaatgccacaagctcctccgcaacagtttccatatcaaccaaattatggaatgggacaacaaccagatccagcctttggtcgagtgtctagtcctcccaatgcaatgatgtcgtcaagaatgggtccctcccagaatcccatgatgcaacacccgcaggctgcatccatctatcagtcctcagaaatgaagggctggccatcaggaaatttggccaggaacagctccttttcccagcagcagtttgcccaccaggggaatcctgcagtgtatagtatggtgcacatgaatggcagcagtggtcacatgggacagatgaacatgaaccccatgcccatgtctggcatgcctatgggtcctgatcagaaatactgctgacatctctgcaccaggacctcttaaggaaaccactgtacaaatgacactgcactaggattattgggaaggaatcattgttccaggcatccatcttggaagaaaggaccagctttgagctccatcaagggtattttaagtgatgtcatttgagcaggactggattttaagccgaagggcaatatctacgtgtttttcccccctccttctgctgtgtatcatggtgttcaaaacagaaatgttttttggcattccacctcctagggatataattctggagacatggagtgttactgatcataaaacttttgtgtcacttttttctgccttgctagccaaaatctcttaaatacacgtaggtgggccagagaacattggaagaatcaagagagattagaatatctggtttctctagttgcagtattggacaaagagcatagtcccagccttcaggtgtagtagttctgtgttgaccctttgtccagtggaattggtgattctgaattgtcctttactaatggtgttgagttgctctgtccctattatttgccctaggctttctcctaatgaaggttttcatttgccattcatgtcctgtaatacttcacctccaggaactgtcatggatgtccaaatggctttgcagaaaggaaatgagatgacagtatttaatcgcagcagtagcaaacttttcacatgctaatgtgcagctgagtgcactttatttaaaaagaatggataaatgcaatattcttgaggtcttgagggaatagtgaaacacattcctggtttttgcctacacttacgtgttagacaagaactatgatttttttttttaaagtactggtgtcaccctttgcctatatggtagagcaataatgctttttaaaaataaacttctgaaaacccaaggccaggtactgcattctgaatcagaatctcgcagtgtttctgtgaatagatttttttgtaaatatgacctttaagatattgtattatgtaaaatatgtatatacctttttttgtaggtcacaacaactcatttttacagagtttgtgaagctaaatatttaacattgttgatttcagtaagctgtgtggtgaggctaccagtggaagagacatcccttgacttttgtggcctgggggaggggtagtgctccacagcttttccttccccaccccccagccttagatgcctcgctcttttcaatctcttaatctaaatgctttttaaagagattatttgtttagatgtaggcattttaattttttaaaaattcctctaccagaactaagcactttgttaatttggggggaaagaatagatatggggaaataaacttaaaaaaaaatcaggaatttaaaaaaacgagcaatttgaagagaatcttttggattttaagcagtccgaaataatagcaattcatgggctgtgtgtgtgtgtgtatgtgtgtgtgtgtgtgtgtatgtttaattatgttaccttttcatcccctttaggagcgttttcagattttggttgctaagacctgaatcccatattgagatctcgagtagaatccttggtgtggtttctggtgtctgctcagctgtcccctcattctactaatgtgatgctttcattatgtccctgtggattagaatagtgtcagttatttcttaagtaactcagtacccagaacagccagttttactgtgattcagagccacagtctaactgagcaccttttaaacccctccctcttctgccccctaccacttttctgctgttgcctctctttgacacctgttttagtcagttgggaggaagggaaaaatcaagtttaattccctttatctgggttaattcatttggttcaaatagttgacggaattgggtttctgaatgtctgtgaatttcagaggtctctgctagccttggtatcattttctagcaataactgagagccagttaattttaagaatttcacacatttagccaatctttctagatgtctctgaaggtaagatcatttaatatctttgatatgcttacgagtaagtgaatcctgattatttccagacccaccaccagagtggatcttattttcaaagcagtatagacaattatgagtttgccctctttcccctaccaagttcaaaatatatctaagaaagattgtaaatccgaaaacttccattgtagtggcctgtgcttttcagatagtatactctcctgtttggagacagaggaagaaccaggtcagtctgtctctttttcagctcaattgtatctgacccttctttaagttatgtgtgtggggagaaatagaatggtgctcttatctttcttgactttaaaaaaattattaaaaacaaaaaaaaaataaatttttttgcaatcctttcctcagacctggctccaggctaactggaaggcagcactcccttttttatatagtagaaaaatgaagtttattataagtttttatattttctacttgttcatttggtgcaaactcaagatttcttttaataggtgcagtctttgagataatttgtttttacctgtattgccctttatcttttttaggtaattctttgtactcctgctgtctacctctcctcacaccccagcaccccccattttttcaaaccttggtatctgttgggtgaacagtataatcttttcatctgcttttagaatgtgggatatttccagtacctactttttttttttttttttgctgaatccaaagatatataaataaaatatatatattttataaagatcagaatgatataaaggagatacatgtttcttcctttaaaaaataaacggaagttacattgttaatgttcatattatgatgccacttttctaaactgcatctggattgaaaggtgtaaatatcaataacagtgctacttagttatcagtatttaatatctgaggtgagttgggggtatctatattaggggtagggtattacagaagataattggcttgatgtcctagaagttctttgatccagaggtgggtgcagctgaaagtaaacagaatggattgccagttacatgtatgcctgcccagttccctttttatttgcagaagctgtgagttttgttcacaattaggttcctaggagcaaaacctcaaggattgatttattgttttcaactccaaggcacactgttaataaacgagcagggtgttttctctcttcctttctaatatatggagtttcgaagaataaaatatgagagcaatatttaaattctcaggaattgacttatactcttgagaatgaattcagtttcaatcaagtttacattatgttgcttaaaaaaatagaaattattctttatcttgcaaagaattgaaaccacatgaaatgacttatgggggatggtgagctgtgactgctttgctgaccattttggatgtcattgtaaataaaggtttctatttaaaattgga

SEQ ID NO: 2 - DNA, 245 bp, Artificial Sequence
  Synthetic primer
tcgactgctgttgaagtgagcgccgttgtcaatatagatacaatagtgaagccacagatgtattgtatctatattgacaacgttgcctactgcctcggaagcagctcactacattactcagctgttgaagtgagcgccgttgtcaatgctgatccaatagtgaagccacagatgtattgtatctatattgacaacgttgcctactgcctcggaagcttaataaaggatcttttattttcattggc

SEQ ID NO: 3 - DNA, 245 bp, Artificial Sequence
  Synthetic primer
tcgactgctgttgaagtgagcgccaaagcaaactcttccgaaatagtgaagccacagatgtatttcggaagagtttgctttgttgcctactgcctcggaagcagctcactacattactcagctgttgaagtgagcgccaaagcaaatacttctgaaatagtgaagccacagatgtatttcggaagagtttgctttgttgcctactgcctcggaagcttaataaaggatcttttattttcattggc

SEQ ID NO: 4 - DNA, 245 bp, Artificial Sequence
  Synthetic primer
tcgactgctgttgaagtgagcgccgttgtcaatatagatacaatagtgaagccacagatgtattgtatctatattgacaacgttgcctactgcctcggaagcagctcactacattactcagctgttgaagtgagcgccgaggaagagagaggtaagttagtgaagccacagatgtaactgacctggttcttcctcgttgcctactgcctcggaagcttaataaaggatcttttattttcattggc

SEQ ID NO: 5 - DNA, 245 bp, Artificial Sequence
  Synthetic primer
tcgactgctgttgaagtgagcgccaaagcaaactcttccgaaatagtgaagccacagatgtatttcggaagagtttgctttgttgcctactgcctcggaagcagctcactacattactcagctgttgaagtgagcgccgaggaagagagaggtaagttagtgaagccacagatgtaactgacctggttcttcctcgttgcctactgcctcggaagcttaataaaggatcttttattttcattggc

SEQ ID NO: 6 - DNA, 245 bp, Artificial Sequence
  Synthetic primer
tcgactgctgttgaagtgagcgcccctatatggtagagcaatatagtgaagccacagatgtatattgctctaccatatagggttgcctactgcctcggaagcagctcactacattactcagctgttgaagtgagcgcccctatatgcatgatcaatatagtgaagccacagatgtatattgctctaccatatagggttgcctactgcctcggaagcttaataaaggatcttttattttcattggc

SEQ ID NO: 7 - DNA, 245 bp, Artificial Sequence
  Synthetic primer
tcgactgctgttgaagtgagcgccggaaatgagatgacagtattagtgaagccacagatgtaatactgtcatctcatttccgttgcctactgcctcggaagcagctcactacattactcagctgttgaagtgagcgccggaaatgactagacactattagtgaagccacagatgtaatactgtcatctcatttccgttgcctactgcctcggaagcttaataaaggatcttttattttcattggc

SEQ ID NO: 8 - DNA, 245 bp, Artificial Sequence
  Synthetic primer
tcgactgctgttgaagtgagcgccatggaaggtacaggaatattagtgaagccacagatgtaatattcctgtaccttccatgttgcctactgcctcggaagcagctcactacattactcagctgttgaagtgagcgccatggaaggactagtaatattagtgaagccacagatgtaatattcctgtaccttccatgttgcctactgcctcggaagcttaataaaggatcttttattttcattggc

SEQ ID NO: 9 - DNA, 245 bp, Artificial Sequence
  Synthetic primer
tcgactgctgttgaagtgagcgcctcatgggaattcatatcattagtgaagccacagatgtaatgatatgaattcccatgagttgcctactgcctcggaagcagctcactacattactcagctgttgaagtgagcgcctcatgggacaacatctcattagtgaagccacagatgtaatgatatgaattcccatgagttgcctactgcctcggaagcttaataaaggatcttttattttcattggc

SEQ ID NO: 10 - DNA, 245 bp, Artificial Sequence
  Synthetic primer
tcgactgctgttgaagtgagcgccccaccaatcagaaacagtatagtgaagccacagatgtatactgtttctgattggtgggttgcctactgcctcggaagcagctcactacattactcagctgttgaagtgagcgccccaccaatgtcaaatagtatagtgaagccacagatgtatactgtttctgattggtgggttgcctactgcctcggaagcttaataaaggatcttttattttcattggc

SEQ ID NO: 11 - DNA, 245 bp, Artificial Sequence
  Synthetic primer
tcgactgctgttgaagtgagcgccggaggagattgatagagcctagtgaagccacagatgtaggctctatcaatctcctccgttgcctactgcctcggaagcagctcactacattactcagctgttgaagtgagcgccggaggagacatatacagcctagtgaagccacagatgtaggctctatcaatctcctccgttgcctactgcctcggaagcttaataaaggatcttttattttcattggc

(19) United States
(12) Patent Application Publication
(10) Pub. No.: US 2013/0266639 A1
(43) Pub. Date: Oct. 10, 2013

(54) METHODS FOR TREATING TRIPLE NEGATIVE BREAST CANCER USING BIFUNCTIONAL SRC 3 shRNA

(71) Applicants: Baylor College of Medicine, Houston, TX (US); Gradalis, Inc., Carrollton, TX (US)

(72) Inventors: Donald Rao, Dallas, TX (US); Zhaohui Wang, Grapevine, TX (US); John J. Nemunaitis, Cedar Hill, TX (US); Neil Senzer, Dallas, TX (US); Bert W. O'Malley, Houston, TX (US); David Lonard, Pearland, TX (US)

(21) Appl. No.: 13/917,499

(22) Filed: Jun. 13, 2013

Related U.S. Application Data
(63) Continuation-in-part of application No. 13/851,464, filed on Mar. 27, 2013.
(60) Provisional application No. 61/616,873, filed on Mar. 28, 2012.

(51) Int. Cl. C12N 15/113; A61N 5/10; A61K 45/06; A61K 31/138; A61K 9/127; A61K 31/713

(52) U.S. Cl. CPC C12N 15/1137; A61K 9/127; A61K 31/713; A61K 45/06; A61K 31/138; A61N 5/10
USPC 424/450; 514/44 A; 424/130.1; 600/1

(57) ABSTRACT

The present invention includes compositions and methods treating triple negative breast cancer comprising administering a therapeutically effective amount of a formulation that includes vector that expresses an SRC-1-specific bifunctional shRNA, an SRC-3-specific bifunctional shRNA, or both, to impair triple negative breast cancer cell growth.

### CROSS-REFERENCE TO RELATED APPLICATIONS

This application claims priority to U.S. Provisional Application Ser. No. 61/616,873, filed Mar. 28, 2012, and is a Continuation-in-Part of U.S. patent application Ser. No. 13/851,464, filed Mar. 27, 2013, the entire contents of each of which are incorporated herein by reference.

### TECHNICAL FIELD OF THE INVENTION

The present invention relates in general to the field of cancer therapy, and more particularly, to a method of treating triple negative breast cancers using a bifunctional SRC-3 shRNA.

### STATEMENT OF FEDERALLY FUNDED RESEARCH

None.

### REFERENCE TO A SEQUENCE LISTING

The present application includes a Sequence Listing which has been submitted in ASCII format via EFS-Web and is hereby incorporated by reference in its entirety. Said ASCII copy, created on Mar. 21, 2013, is named GRAD:1036.txt and is 16 KB in size.

### BACKGROUND OF THE INVENTION

Without limiting the scope of the invention, its background is described in connection with breast cancer and SRC-3 RNA interference.

U.S. Pat. No. 7,282,576 (Riegel et al. 2007) relates to AIB1 amplification in a subset of human breast cancers and the appearance of a splice variant that lacks exon-3 and causes the protein to lack the N-terminal basic-loop-basic domain in breast cancer cells.

U.S. Patent Application No. 2007/0099209 (Clarke et al. 2007) relates to compositions and methods for treating, characterizing, and diagnosing cancer and provides gene expression profiles associated with solid tumor stem cells.

U.S. Patent Application Publication No 2010/0286244 (Addepalli et al., 2010) relates to the use of short interfering nucleic acid molecules (siRNA) to inhibit Nuclear Mitotic Apparatus Protein (NuMA) gene expression and their use in treatment of disease, including cancer.

U.S. Patent Application Publication No. 2004/0086911 (Cabello et al. 2004) is directed to RNA interference using novel compositions and methods. In particular embodiments, the RNA compositions comprise double strand regions interrupted with non-complementary regions, wherein the RNA compositions are effective for regulation of transcription. In specific embodiments, transcription of a target nucleic acid sequence to which the RNA composition is directed is reduced or inhibited, such as by inducing destruction of at least one transcript. In other embodiments, multiple target nucleic acid sequences are targeted by the RNA compositions of the present invention.

### SUMMARY OF THE INVENTION

Disclosed are methods and compositions relating to SRC-3 bifunctional shRNA employed and the treatment of cancer. In one embodiment, the present invention includes a method for treating triple negative breast cancer comprising administering a therapeutically effective amount of a formulation that includes vector that expresses an SRC-1-specific bifunctional shRNA, an SRC-3-specific bifunctional shRNA, or both, to impair triple negative breast cancer cell growth. In one aspect, the formulation further comprises a cationic liposomal preparation. In another aspect, the cationic liposomal preparation comprises a single vector that encodes the SRC-1-specific bifunctional shRNA, the SRC-3-specific bifunctional shRNA, or both the SRC-1-specific bifunctional shRNA and the SRC-3-specific bifunctional shRNA. In another aspect, the one or more shRNA's is comprises a sequence selected from SEQ ID NO: 2, SEQ ID NO: 3, SEQ. ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or combinations or modifications thereof. In another aspect, a sequence arrangement for the shRNA comprises a 5′ stem arm-19 nucleotide target (SRC-3 gene)-TA-15 nucleotide loop-19 nucleotide target complementary sequence-3′ stem arm-Spacer-5′ stem arm-19 nucleotide target variant-TA-15 nucleotide loop-19 nucleotide target complementary sequence-3′ stem arm. In another aspect, the one or more polycations is a 10 kDA polyethylene glycol (PEG)-substituted cysteine-lysine 3-mer peptide (CK30PEG10k). In another aspect, the compacted DNA nanoparticles are further encapsulated in a liposome. In another aspect, the liposome is a bilamellar invaginated vesicle (BIV). In another aspect, the triple negative breast cancer is resistant to chemotherapeutic agents.

Another embodiment of the present invention includes a method of treating a triple negative breast cancer in a human subject comprising the steps of: identifying a human subject in need for suppression of triple negative breast cancer cell growth; and administering an expression vector in a therapeutic agent carrier complex to the human subject in an amount sufficient to suppress the growth of a triple negative breast cancer cells; wherein the expression vector expresses one or more bifunctional short hairpin RNA (shRNA) capable inhibiting an expression of an SRC-1 gene, an SRC-3 gene, or both, wherein the one or more shRNA comprise a bifunctional RNA molecule that activates concurrently both a cleavage-dependent and a cleavage-independent RNA-induced silencing complex for reducing the expression level of the SRC-1 gene, the SRC-3 gene, or both. In one aspect, the one or more shRNAs are selected from the group consisting of SEQ ID NO: 2, SEQ ID NO: 3, SEQ. ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and combinations or modifications thereof. In another aspect, a sequence arrangement for the shRNA comprises a 5′ stem arm-19 nucleotide target (SRC-3 gene)-TA-15 nucleotide loop-19 nucleotide target complementary sequence-3′ stem arm-Spacer-5′ stem arm-19 nucleotide target variant-TA-15 nucleotide loop-19 nucleotide target complementary sequence-3′ stem arm. In another aspect, the therapeutic agent carrier is a compacted DNA nanoparticle or a reversibly masked liposome decorated with one or more receptor targeting moieties, wherein the one or more receptor targeting moieties are small molecule bivalent beta-turn mimics. In another aspect, the therapeutic agent carrier is a compacted DNA nanoparticle that is compacted with one or more polycations, wherein the one or more polycations comprises a 10 kDA polyethylene glycol (PEG)-substituted cysteine-lysine 3-mer peptide (CK30PEG10k) or a 30-mer lysine condensing peptide. In another aspect, the reversibly masked liposome is a bilamellar invaginated vesicle (BIV). In another aspect, the compacted DNA nanoparticles are further encapsulated in a liposome. In another aspect, the tumor cell or breast cancer is resistant to tamoxifen therapy. In another aspect, the method further comprises the step of administering tamoxifen. In another aspect, the method further comprises the step of administering the vector before, after, or concurrently as a combination therapy with one or more treatment methods selected from the group consisting of chemotherapy, radiation therapy, surgical intervention, antibody therapy, Vitamin D therapy, or any combinations thereof. In another aspect, the triple negative breast cancer is resistant to chemotherapeutic agents.

Yet another embodiment of the present invention includes a method of treating one or more cancers resistant to chemotherapy, increasing effectiveness of one or more chemotherapeutic agents, or both in a subject comprising the steps of: identifying the human or animal subject having the cancer resistant to the chemotherapeutic agents or in need of increased effectiveness of the one or more chemotherapeutic agents, wherein the breast cancer is a triple negative breast cancer; and administering an expression vector in a therapeutic agent carrier complex to the human or animal subject in an amount sufficient to suppress or inhibit an expression of an SRC-1 gene, an SRC-3 gene, or both in the subject, wherein the expression vector expresses one or more bifunctional short hairpin RNA (shRNA) capable inhibiting the expression of the SRC-1 gene, the SRC-3 gene, or both in one or more triple negative breast cancer cells in the subject via RNA interference, wherein the inhibition results in an enhanced action of the one or more chemotherapeutic agents leading to an apoptosis, an arrested proliferation, or a reduced invasiveness of one or more triple negative breast cancer cells; wherein the one or more bifunctional shRNA activate a cleavage-dependent and a cleavage-independent RNA-induced silencing complex for reducing the expression level of SRC-1, SRC-3, or both. In one aspect, the one or more chemotherapeutic agents comprise platinum drugs, carboplatin, tamoxifen, ER antagonists, or any combinations thereof. In another aspect, the cancers are selected from the group consisting of colon, breast, pancreatic, prostate, or any combinations thereof. In another aspect, the one or more shRNAs are selected from the group consisting of SEQ ID NO: 2, SEQ ID NO: 3, SEQ. ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and combinations or modifications thereof. In another aspect, the vector is administered before, after, or concurrently as with the one or more chemotherapeutic agents. In another aspect, the triple negative breast cancer is resistant to chemotherapeutic agents.

In one embodiment the present invention discloses an expression vector comprising: a promoter and a nucleic acid insert operably linked to the promoter, wherein the insert encodes one or more short hairpin RNAs (shRNA) capable of inhibiting an expression of a SRC-3 gene via RNA interference. The shRNA described herein incorporates one or more siRNA (cleavage-dependent) and miRNA (cleavage-independent) motifs. Furthermore, the shRNA is both the cleavage-dependent and cleavage-independent inhibitor of the expression of the SRC-3 gene. In one aspect the shRNA is further defined as a bifunctional shRNA. In another aspect the one or more shRNA's that inhibit the SRC-3 gene is selected from the group consisting of SEQ ID NO: 2, SEQ ID NO: 3, SEQ. ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and combinations or modifications thereof.

Another embodiment herein discloses a therapeutic delivery system comprising: a therapeutic agent carrier and an expression vector comprising a promoter and a nucleic acid insert operably linked to the promoter, wherein the insert encodes one or more short hairpin RNA (shRNA) capable of inhibiting an expression of a SRC-3 gene via RNA interference.

In one aspect the therapeutic agent carrier is a compacted DNA nanoparticle, wherein the DNA nanoparticle is compacted with one or more polycations. In a specific aspect the one or more polycations is a 10 kDA polyethylene glycol (PEG)-substituted cysteine-lysine 3-mer peptide (CK₃₀PEG10k). In another aspect the compacted DNA nanoparticles are further encapsulated in a liposome, wherein the liposome is a bilamellar invaginated vesicle (BIV) or a reversibly masked liposome. In yet another aspect the liposome is decorated with one or more “smart” receptor targeting moieties, wherein the one or more “smart” receptor targeting moieties are small molecule bivalent beta-turn mimics.

In one aspect the therapeutic agent carrier is a liposome. In another aspect the liposome is a bilamellar invaginated vesicle (BIV) decorated with one or more “smart” receptor targeting moieties, wherein the liposome is a reversibly masked liposome, wherein the “smart” receptor targeting moieties are small molecule bivalent beta-turn mimics. In yet another aspect the one or more shRNA's that inhibit the SRC-3 gene are selected from the group consisting of SEQ ID NO: 2, SEQ ID NO: 3, SEQ. ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and combinations or modifications thereof. In another aspect the delivery system is used to suppress tumor cell growth, treat breast cancer, or both by itself or in combination with one or more chemotherapeutic agents, radiation therapy, surgical intervention, antibody therapy, Vitamin D, or any combinations thereof.

Yet another embodiment disclosed herein relates to a method to deliver one or more shRNAs to a target tissue expressing a SRC-3 gene, comprising the steps of: (i) preparing an expression vector comprising a promoter and a nucleic acid insert operably linked to the promoter that encodes the one or more shRNAs inhibits an expression of a SRC-3 gene, via RNA interference; (ii) combining the expression vector with a therapeutic agent carrier, wherein the therapeutic agent carrier is a liposome decorated with one or more “smart” receptor targeting moieties; and (iii) administering a therapeutically effective amount of the expression vector and therapeutic agent carrier complex to a patient in need thereof.

The present invention further provides a method to inhibit an expression of a SRC-3 gene in one or more target cells comprising the steps of: selecting the one or more target cells; and transfecting the target cell with a vector that expresses one or more short hairpin RNA (shRNAs) capable of inhibiting an expression of a SRC-3 gene in the one or more target cells via RNA interference.

Another embodiment disclosed herein is a method of suppressing a tumor cell growth, treating breast cancer, or both in a human subject comprising the steps of: identifying the human subject in need for suppression of the tumor cell growth, treatment of breast cancer or both; and administering an expression vector in a therapeutic agent carrier complex to the human subject in an amount sufficient to suppress the tumor cell growth, treat breast cancer or both, wherein the expression vector expresses one or more bifunctional short hairpin RNA (shRNA) capable inhibiting an expression of a SRC-3 gene in the one or more target cells via RNA interference, wherein the inhibition results in an apoptosis, an arrested proliferation, or a reduced invasiveness of the tumor cells.

In yet another embodiment the present invention provides a method of treating one or more cancers resistant to chemotherapy, increasing effectiveness of one or more chemotherapeutic agents, or both in a human or animal subject comprising the steps of: identifying the human or animal subject having the cancer resistant to the chemotherapeutic agents or in need of increased effectiveness of the one or more chemotherapeutic agents and administering an expression vector in a therapeutic agent carrier complex to the human or animal subject in an amount sufficient to suppress or inhibit an expression of a SRC-3 gene in the human or the animal subject, wherein the expression vector expresses one or more bifunctional short hairpin RNA (shRNA) capable inhibiting the expression of a SRC-3 gene in one or more target cells in the human or animal subject via RNA interference, wherein the inhibition results in an enhanced action of the one or more chemotherapeutic agents leading to an apoptosis, an arrested proliferation, or a reduced invasiveness of one or more tumor cells.

In one aspect the one or more chemotherapeutic agents comprise platinum drugs, carboplatin, tamoxifen, ER antagonists, or any combinations thereof. In another aspect the cancers are selected from the group consisting of colon, breast, pancreatic, prostate, or any combinations thereof. In a specific aspect the cancer is HER-2 positive breast cancer. In yet another aspect the one or more shRNAs are selected from the group consisting of SEQ ID NO: 2, SEQ ID NO: 3, SEQ. ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and combinations or modifications thereof. In a related aspect the vector is administered before, after, or concurrently as with the one or more chemotherapeutic agents.

A method of treating chemotherapy resistant HER-2 positive breast cancer, increasing effectiveness or a chemotherapeutic regimen in HER-2 positive breast cancer, or both in a human or animal subject is provided in one embodiment of the present invention. The method as described herein comprises the steps of: identifying the human or animal subject suffering from the chemotherapy resistant HER-2 positive breast cancer or needing increased effectiveness of the chemotherapy against HER-2 positive breast cancer and administering an expression vector in a therapeutic agent carrier complex to the human or animal subject in an amount sufficient to suppress or inhibit an expression of a SRC-3 gene in the human or the animal subject, wherein the expression vector expresses one or more bifunctional short hairpin RNA (shRNA) capable inhibiting the expression of a SRC-3 gene in one or more target cells in the human or animal subject via RNA interference, wherein the inhibition results in an enhanced action of the one or more chemotherapeutic agents leading to an apoptosis, an arrested proliferation, or a reduced invasiveness of one or more tumor cells. In one aspect of the method the one or more chemotherapeutic agents comprise platinum drugs, carboplatin, tamoxifen, ER antagonists, or any combinations thereof.

### BRIEF DESCRIPTION OF THE DRAWINGS

For a more complete understanding of the features and advantages of the present invention, reference is now made to the detailed description of the invention along with the accompanying figures.

FIG. 1 depicts mechanism of coactivator-targeting agents that have the potential to be more effective anti-cancer drugs;

FIG. 2 shows that SRC-3 overexpression is associated with resistance to carboplatin therapy in human ovarian cancer, X-axis difference cell lines, Y-axis relative expression of SRC-3;

FIG. 3 documents that three bishSRC-3 vectors (pGB145, 48, and 49) can effectively block SRC-3 protein expression in T-47D breast cancer cells. BishSRC-3 vectors (L4-L6), Dharmacon siRNA SMART pool (L8, and L9) and its non-targeting control (L7) and the bishSRC-3 empty vector pUMVC3 (L3) were introduced into cells via electroporation and assayed for SRC-3 protein levels 72 hours later;

FIGS. 4A and 4B are schematic representations showing the design of the bi-functional shRNAs of the present invention. FIG. 4A shows the sequence arrangement for a single target, and FIG. 4B shows the sequence arrangement for multiple targets;

FIGS. 5A-5F are plasmid maps for the different bi-shRNA-NCOA3 of the present invention;

FIG. 6 shows that a SRC-3 targeting bifunctional shRNAs can reduce SRC-3 protein expression in MCF-7 breast cancer cells. bishRNA vectors (pGBI-45-pGBI-49), the empty parent vector pUMCV3 (Vec) and siRNA as a positive control (siSRC-3) were ‘reverse’ transfected into MCF-7 cells. 1 μg DNA/lipofectamine complexes were added to 6 well plates followed by plating of MCF-7 cells. 48 hours after cell plating, protein extracts were analyzed by Western blotting;

FIG. 7 shows that SRC-3 targeting bifunctional shRNAs can reduce SRC-3 protein expression in MDA-MB-231 breast cancer cells. bishRNA vectors (pGBI-45-pGBI-49), the empty parent vector pUMCV3 (Vec) and siRNA as a positive control (siSRC-3) were ‘reverse’ transfected into MCF-7 cells. 1 μg DNA/lipofectamine complexes were added to 6 well plates followed by plating of MDA-MB-231 cells. 48 hours after cell plating, protein extracts were analyzed by Western blotting;

FIG. 8 shows that SRC-1 and SRC-3 bi-shRNA vectors impair breast cancer cell growth over a four day period. MCF-7 cells were transfected with bi-shRNA vectors for SRC-1 (pGBI40-pGBI-44) or SRC-3 (pGBI-45-pGBI-49), or siRNA negative control (siGFP) or positive control (siSRC-3) as described for FIGS. 6 and 7. Four days after later, cell proliferation was measured by MTS assay;

FIG. 9 shows that SRC-1 and SRC-3 bi-shRNA vectors impair breast cancer cell growth over a five day period. MCF-7 cells were transfected by bi-shRNA vectors for SRC-1 (pGBI-40-pGBI-44) or SRC-3 (pGBI-45-pGBI-49), or siRNA negative control (siGFP) or positive control (siSRC-3) as described for FIGS. 6 and 7. Five days after later, call proliferation was measured by MTS assay;

FIG. 10 shows that SRC-1 and SCR-3 bi-shRNA vectors impair triple negative breast cancer cell growth over a four day period. MCDA-MB-231 cells were transfected with SRC-1 (pGBI-40-pGBI-44) or SRC-3 (pGBI-45-pGBI-49) or empty vector control. Four days after, cell proliferation was measured by MTS assay; and

FIG. 11 shows that SRC-1 and SRC-3 targeting bifunctional shRNAs can reduce SRC-3 protein expression in A-549 lung cancer cells. Bi-shRNA vectors for SRC-1 (pGBI-40-pGBI-44), or SRC-3 (pGBI-45-pGBI-49), or empty vector control were transected into A-549 cells. 48 hrs post-transfection, SRC-3 expression was monitored by western immunoblot.

FIG. 12 shows that five different bishSRC-3 vectors can reduce SRC-3 protein expression in MCF-7 and MDA-231 breast cancer cells. bishSRC-3 vectors (pGBI 45-49) or their parent, empty expression vector pUMVC3 (Vec) or a Dharmacon SRC-3 targeting siRNA pool (siSRC-3) were reversed transfected into cells and assayed for SRC-3 protein levels 72 hours later.

FIG. 13 shows that five different bishSRC-3 vectors can reduce SRC-3 protein expression in MCF-7 and MDA-231 breast cancer cells. Untreated (Un), bishSRC-3 vectors (pGBI 45-49) or their parent, empty expression vector pUMVC3 (Vec) were reversed transfected into cells and assayed for SRC-3 protein levels 72 hours later. Cell proliferation was measured by MTS assay.

FIG. 14 is a graph that shows that SRC-3 targeting bifunctional shRNA vector pGBI-45 suppresses the primary tumor growth in the mouse LM3 (MDA-MD-231 subline) xenograft model system. Tumor bearing mice were treated once a week with water control (D5W) or SRC-3 bishRNA (pGBI-45) via tail vein injection. Tumor volume was measured on indicated days after initial treatment.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “expression vector” as used herein in the specification and the claims includes nucleic acid molecules encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter. The term “promoter” refers to any DNA sequence which, when associated with a structural gene in a host yeast cell, increases, for that structural gene, one or more of 1) transcription, 2) translation or 3) mRNA stability, compared to transcription, translation or mRNA stability (longer half-life of mRNA) in the absence of the promoter sequence, under appropriate growth conditions.

The term “oncogene” as used herein refers to genes that permit the formation and survival of malignant neoplastic cells (Bradshaw, T. K.: Mutagenesis 1, 91-97 (1986).

As used herein the term “receptor” denotes a cell-associated protein that binds to a bioactive molecule termed a “ligand.” This interaction mediates the effect of the ligand on the cell. Receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor). Membrane-bound receptors are characterized by a multi-domain structure comprising an extracellular ligand-binding domain and an intracellular effector domain that is typically involved in signal transduction. In certain membrane-bound receptors, the extracellular ligand-binding domain and the intracellular effector domain are located in separate polypeptides that comprise the complete functional receptor.

The term “hybridizing” refers to any process by which a strand of nucleic acid binds with a complementary strand through base pairing.

The term “transfection” refers to the introduction of foreign DNA into eukaryotic cells. Transfection may be accomplished by a variety of means known to the art including, e.g., calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

As used herein the term “bi-functional” refers to a shRNA having two mechanistic pathways of action, that of the siRNA like (loading of pre-shRNA onto cleavage-dependent RISCs) and that of an miRNA-like moiety (loading of pre-shRNA onto cleavage-independent RISCs with complete target mRNA complementarity). A bifunctional construct concurrently represses the translation of the target mRNA, facilitates mRNA degradation and p-body mRNA sequestration, and cleaves target mRNA through RNAse H-like cleavage.

The term “traditional” shRNA refers to a DNA transcription derived RNA acting by the siRNA mechanism of action. The term “doublet” shRNA refers to two shRNAs, each acting against the expression of two different genes but in the “traditional” siRNA mode.

As used herein, the term “liposome” refers to a closed structure composed of lipid bilayers surrounding an internal aqueous space. The term “polycation” as used herein denotes a material having multiple cationic moieties, such as quaternary ammonium radicals, in the same molecule and includes the free bases as well as the pharmaceutically-acceptable salts thereof.

As used herein, the phrase “triple negative breast cancer” refers to those breast cancer cells that are negative for estrogen (ER), progesterone (PR) and HER2/neu (HER2) receptors. The “triple negative” status for breast cancer cells is generally associated with a poor prognosis in early breast cancer patients. The term “triple negative breast cancer” is often used interchangeably or as a clinical surrogate for “basal-like” breast cancers. In one specific embodiment of the present invention the breast cancer cells, whether phenotypic or genotypic triple negative, or

The present inventors appreciate that there are a number of strategies for targeting a protein interaction domain. For example, it may be possible to find a small molecule that inhibits the protein/protein interaction; this would require a high throughput assay and extensive screening followed by optimization of the compound for clinical applications likely requiring several years of effort. An alternative would be to prepare a cell permeable peptide derivative, but the quantities needed for clinical applications likely would be impractical. Thus, the present invention includes embodiments in which a plasmid Lipoplex combination is used to deliver an expression plasmid that will produce the peptide that inhibits AR action.

In one embodiment of the present disclosure, the delivery vehicle comprises DNA encapsulated in cationic bilamellar invaginated vesicles (BIV). In another embodiment, 200-450 nm BIV are prepared from cholesterol and biodegradable 1,2-dioleoyl-3-trimethyl-ammonio-propane (DOTAP). In yet another embodiment, the positive charge is reversibly masked (rm) by the addition of a neutral small molecular weight lipid such as dodecyl-βmaltopyranoside, which prevents initial non-specific uptake.

The present inventors recognize that delivery vehicle BIV/plasmid lipoplex can penetrate tumor capillaries and are taken up by fusion, minimizing degradation of DNA; that they give higher levels of gene expression when injected in mice than other methods of delivery, and that this technology can be used to express transgenes in humans in the target tissue without major toxicity.

In a preferred embodiment, two or more approaches of treatment are combined to provide a more effective treatment than either alone.

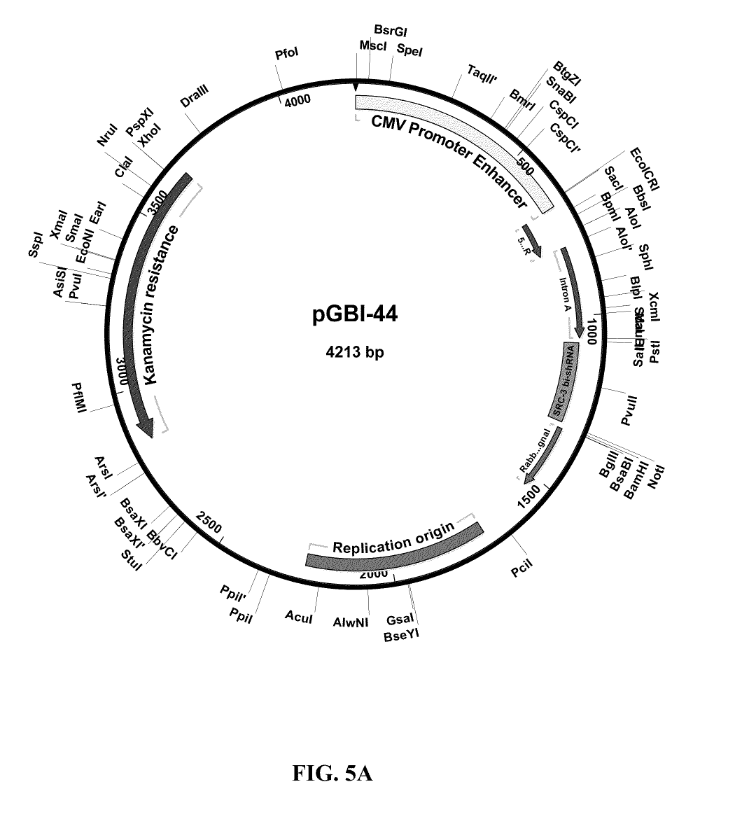

The present inventors have pioneered a unique RNAi platform known as bi-functional shRNA. Conceptually, RNAi can be achieved through shRNA-loaded RISCs to promote cleavage-dependent or cleavage-independent mRNA knockdown. Concomitant expression of both configurations of shRNAs (hence the nomenclature, bi-functional shRNA) to promote loading onto multiple types of RISCs has been shown by the present inventors to achieve more effective target gene knockdown at a more rapid onset of silencing (rate of mRNA and protein turnover notwithstanding) with greater durability as compared with siRNA. The basic design of the bi-functional shRNA expression unit comprises two stem-loop shRNA structures; one composed of fully matched passenger and guide strands for cleavage-dependent RISC loading, and a second stem-loop with a mismatched passenger strand (at positions 9-12) for cleavage-independent RISC loading. This bi-functional design is, much more efficient for two reasons; first, the bi-functional promotes guide strand loading onto distinct RISC types, hence promoting mRNA targeting; second, the presence of cleavage-dependent and cleavage-independent RISCs against the same target mRNA promotes silencing by both degradation and translational inhibition/sequestration processes. The potent gene knockdown effector achieves spatial and temporal control by the multiplexed shRNAs under the control of a single pol II promoter. The platform designed by the present inventors mimics the natural process. Multiple studies by others and the literature support the approach of the present inventors. A schematic representation of the bi-functional shRNA design against a single or against multiple targets is shown in FIGS. 4A and 4B, respectively.

Liposomal delivery system: The liposomal delivery system involves 1,2-dioleoyl-3-trimethyl-ammoniopropane (DOTAP) and cholesterol. This formulation combines with DNA to form complexes that encapsulate nucleic acids within bilamellar invaginated vesicles (liposomal BIVs). Applicants have optimized several features of the BIV delivery system for improved delivery of RNA, DNA, and RNAi plasmids. The liposomal BIVs are fusogenic, thereby bypassing endocytosis mediated DNA cell entry, which can lead to nucleic acid degradation and TLR mediated off-target effects.

The present inventors recognize that an optimized delivery vehicle needs to be a stealthed, which can achieved by PEGylation of nanoparticle with a zeta potential of ≦10 mV for efficient intravascular transport in order to minimize nonspecific binding to negatively-charged serum proteins such as serum albumin (opsonization). Incorporation of targeting moieties such as antibodies and their single chain derivatives (scFv), carbohydrates, or peptides may further enhance transgene localization to the target cell.

The present inventors have created targeted delivery of the complexes in vivo without the use of PEG thereby avoiding an excessively prolonged circulatory half-life. While PEGylation is relevant for DNA or siRNA oligonucleotide delivery to improve membrane permeability, the present inventors recognize that the approach may cause steric hindrance in the BIV liposomal structures, resulting in inefficient DNA encapsulation and reduced gene expression. Furthermore, PEGylated complexes enter the cell predominantly through the endocytic pathway, resulting in degradation of the bulk of the nucleic acid in the lysosomes. While PEG provides extremely long half-life in circulation, this has created problems for patients as exemplified by doxil, a PEGylated liposomal formulation that encapsulates the cytotoxic agent doxorubicin. Attempts to add ligands to doxil for delivery to specific cell surface receptors (e.g. HER2/neu) have not enhanced tumor-specific delivery.

The present disclosure includes embodiments in which BIVs are produced with DOTAP, and synthetic cholesterol using proprietary manual extrusion process. Furthermore, the delivery was optimized using reversible masking technology. Reversible masking utilizes small molecular weight lipids (about 500 Mol. Wt. and lower; e.g. n-dodecyl-β-D-maltopyranoside) that are uncharged and, thereby, loosely associated with the surface of BIV complexes, thereby temporarily shielding positively charged BIV complexes to bypass non-targeted organs. These small lipids are removed by shear force in the bloodstream. By the time they reach the target cell, charge is re-exposed (optimally ˜45 mV) to facilitate entry.

One reason that the BIV delivery system is uniquely efficient is because the complexes deliver therapeutics into cells by fusion with the cell membrane and avoid the endocytic pathway. The two major entry mechanisms of liposomal entry are via endocytosis or direct fusion with the cell membrane. The inventors found that nucleic acids encapsulated in BIV complexes delivered both in vitro and in vivo enter the cell by direct fusion and that the BIVs largely avoid endosomal uptake, as demonstrated in a comparative study with polyethylene-amine (PEI) in mouse alveolar macrophages. PEI is known to be rapidly and avidly taken up into endosomes, as demonstrated by the localization of ≧95% of rhodamine labeled oligonucleotides within 2-3 hrs post-transfection.

Cancer targeted delivery with decorated BIVs: The present inventors recognize that siRNAs that are delivered systemically by tumor-targeted nanoparticles (NPs) are significantly more effective in inhibiting the growth of subcutaneous tumors, as compared to undecorated NPs. Targeted delivery does not significantly impact pharmacokinetics or biodistribution, which remains largely an outcome of the EPR (enhanced permeability and retention) effect, but appears to improved transgene expression through enhanced cellular uptake [95-97].

Indeed, a key “missing piece” in development of BIVs for therapeutic is the identification of such non-immunogenic ligands that can be placed on the surface of BIV-complexes to direct them to target cells. While it might be possible to do this with small peptides that are multimerized on the surface of liposomes, these can generate immune responses after repeated injections. Other larger ligands including antibodies, antibody fragments, proteins, partial proteins, etc. are far more refractory than using small peptides for targeted delivery on the surface of liposomes. The complexes of the present invention are thus unique insofar as they not only penetrate tight barriers including tumor vasculature endothelial pores and the interstitial pressure gradient of solid tumors, but also target tumor cells directly. Therefore, the therapeutic approach of the present invention is not limited to delivery solely dependent on the EPR effect but targets the tumor directly.

Small molecules designed to bind proteins selectively can be used with the present invention. Importantly, the small molecules prepared are “bivalent” so they are particularly appropriate for binding cell surface receptors, and resemble secondary structure motifs found at hot-spots in protein-ligand interactions. The present inventors have adapted a strategy to give bivalent molecules that have hydrocarbon tails, and prepared functionalized BIV complexes from these adapted small molecules. An efficient high throughput technology to screen the library was developed and run.

Compacted DNA Nanoparticles: Safe and Efficient DNA Delivery in Post-Mitotic Cells:

The Copernicus nucleic acid delivery technology is a non-viral synthetic and modular platform in which single molecules of DNA or siRNA are compacted with polycations to yield nanoparticles having the minimum possible volume. The polycations optimized for in vivo delivery is a 10 kDa polyethylene glycol (PEG) modified with a peptide comprising a N-terminus cysteine and 30 lysine residues (CK₃₀PEG10k). The shape of these complexes is dependent in part on the lysine counterion at the time of DNA compaction. The minimum cross-sectional diameter of the rod nanoparticles is 8-11 nm irrespective of the size of the payload plasmid, whereas for ellipsoids the minimum diameter is 20-22 nm for typical expression plasmids. Importantly, these DNA nanoparticles are able to robustly transfect non-dividing cells in culture. Liposome mixtures of compacted DNA generate over 1,000-fold enhanced levels of gene expression compared to liposome naked DNA mixtures. Following in vivo dosing, compacted DNA robustly transfects post-mitotic cells in the lung, brain, and eye. In each of these systems the remarkable ability of compacted DNA to transfect post-mitotic cells appears to be due to the small size of these nanoparticles, which can cross the cross the 25 nm nuclear membrane pore.

One uptake mechanism for these DNA nanoparticles is based on binding to cell surface nucleolin (26 nm K_{D}), with subsequent cytoplasmic trafficking via a non-degradative pathway into the nucleus, where the nanoparticles unravel releasing biologically active DNA. Long-term in vivo expression has been demonstrated for as long as 1 year post-gene transfer. These nanoparticles have a benign toxicity profile and do not stimulate toll-like receptors thereby avoiding toxic cytokine responses, even when the compacted DNA has hundreds of CpG islands and are mixed with liposomes, no toxic effect has been observed [114,115]. DNA nanoparticles have been dosed in humans in a cystic fibrosis trial with encouraging results, with no adverse events attributed to the nanoparticles and with most patients demonstrating biological activity of the CFTR protein [116].

The construction of a novel bi-shRNA therapeutic of the present invention represents a state-of the art approach that can reduce the effective systemic dose needed to achieve an effective therapeutic outcome through post-transcriptional gene knockdown. Effective and clinically applicable delivery approaches are in place that can be rapidly transitioned for systemic targeting of ESFTs.

The present invention describes an innovative bifunctional shRNAs-based strategy designed to achieve superior knockdown of steroid receptor coactivator-3 (SRC-3). These bifunctional shRNAs achieve their enhanced function by simultaneously promoting target mRNA degradation and translational repression. SRC-3 is amplified in breast cancer is a key breast cancer oncogene that is frequently overexpressed or amplified in estrogen receptor-α (ERα) and HER2 positive breast cancers; that elevated expression of SRC-3 is associated with resistance to tamoxifen therapy and with poor disease outcome in HER2 positive breast cancers.

The present inventors also recognize that targeting of SRC-3 limits breast cancer cell growth and restores the anti-estrogenic actions of tamoxifen. The present inventors appreciate that chemotherapeutic agents that target both ERα and HER2 have been extensively pursued and developed and both their effectiveness and limitations are well characterized.

The present disclosure includes embodiments in which bifunctional small hairpin RNAs (bishRNAs), targeting SRC-3 are employed as therapeutic agents against tamoxifen and HER2 resistant breast cancers. One way to evaluate the ability of these SRC-3 bishRNAs (bishSRC-3s) to block cell growth and resistance to tamoxifen and anti-HER2 treatment is to use Tamoxifen and anti-HER2 sensitive and resistant breast cancer cell lines.

The present inventors recognize the central role that SRC-3 plays in breast cancers as well as the lack of clinically available agents to target SRC-3, and that the present disclosure represents a unique approach to characterize and develop a unique class of breast cancer chemotherapeutic agents.

The present inventors recognize that SRC-3 bishRNAs (bishSRC-3s) that is designed to block expression of the oncogenic coactivator SRC-3 is able to block breast cancer cell growth and is able to overcome tamoxifen and anti-HER2 chemotherapeutic resistance. One way to demonstrate this outcome is to use cell culture and animal model systems.

The present inventors also appreciate the importance of determining the pleiotropic impact of SRC-3 bishRNAs on cancer cell growth pathways and its effectiveness in a cell culture-based breast cancer combination chemotherapy paradigm. One way to define how different bishSRC-3s influence global gene expression patterns responsible for cancer cell growth is by mRNA microarray analysis in ERα-positive, ERα-positive tamoxifen resistant, and HER2-positive breast cancer cells. To functionally assess bishSRC-3s' ability to abrogate chemotherapy resistance, the bishSRC-3s are incorporated in a combination chemotherapy model paradigm using tamoxifen- and herceptin-resistant breast cancer cell culture systems.

One way of evaluating and assess SRC-3 bishRNAs in preclinical breast cancer chemotherapy resistance animal model systems is to use human tumor cell-mouse host xenograft models (evaluating effectiveness of bishSRC-3 in blocking xenograft tumor transplants from tamoxifen-sensitive, tamoxifen-resistant, and anti-HER2 resistant breast cancers. A way to enhance delivery to tumor tissue is to use of a novel lipoplex formulation developed.

The present inventor recognize that breast cancer is the second most common form of cancer and is responsible for 7% of all cancer deaths (according to American Cancer Society). Survival rates for patients with primary breast cancer vary based on a variety of factors, among them breast cancer subtype, nuclear receptor status (ERα, progesterone receptor (PR)), HER2/neu status, and transcriptome expression patterns.

The inventors appreciate that ERα, PR and HER2/neu are key prognostic and diagnostic markers for directing clinical interventions for breast cancer treatment (1). While progress has been made in the treatment of ERα(+) as well as HER2(+) disease, many patients still recur and die after these patients' tumors acquire resistance to established chemotherapies (2).

One of the present inventors' major goal is also to “personalize” the therapy of individual patients based on tumor biomarkers or pharmacogenomic considerations to develop the best possible treatment for each individual patient (3). The present inventors recognize the limited ability of any one therapeutic target or strategy by itself to block cancer cell growth and appreciate the benefit of approaches that use a combination of targets and strategies to improve the efficacy of anti-cancer agents for chemotherapy-resistant breast cancers (4). The present inventors also recognize that a growing body of data points to the fact that nuclear receptor coactivators are key drivers of cancer cell growth (5).

The present inventors recognize steroid receptor coactivator-3/Amplified in breast cancer 1 (SRC-3) as a key oncogenic coactivator in breast cancers that plays a driving role in breast cancer acquisition of resistance to tamoxifen and anti-HER2 therapies; and that SRC-3 is a suitable target for a coactivator-targeting agent.

The present inventor also recognize that SRC-3 is a key oncogenic nuclear receptor coactivator; that nuclear hormone receptor coactivators are required for nuclear receptors to function as transcription factors and play key roles as rheostats that determine the amplitude of biological responses to steroid hormones (6); that overexpression of the steroid receptor coactivator-3/amplified in breast cancer 1 (SRC-3) is implicated in a wide range of cancers and is frequently overexpressed at high percentages in hormone-dependent cancers such as breast, ovarian (2), endometrial (7) and prostate cancers (4), and other cancers including pancreatic (8), esophageal (9), nasopharyngeal (10), urothelial (6) and colorectal cancers (7). The present inventors appreciate that in breast and ovarian cancers where it was first characterized, the SRC-3 gene is amplified in approximately 10% of breast cancers and its mRNA is overexpressed ˜64% of the time (8); and that elevated expression of SRC-3 also has been associated with resistance to tamoxifen therapy and poor disease outcome (11).

TABLE 1
Relationship of SRC-3 to selected major cancers
incidences and deaths in the United States

| | New | Deaths | % SRC-3 | |
|---|---|---|---|---|
| Cancer Type | cases | per year | overex. | Reference |
| Colon | 108,000 | 57,000 | 36.5% | Xie et al. 2005 |
| Breast | 182,000 | 40,000 | 64% | Anzick et al., 1997 |
| Prostate | 186,000 | 29,000 | 76% | Gnanapragasam |
| | | | | et al., 2001 |
| Pancreatic | 38,000 | 34,000 | 65% | Henke et al., 2004 |

At least 322,000 new SRC-3 related cancer cases and 91,000 SRC-3 cancer related deaths are predicted to occur yearly in the United States (restricted to the above cancer types). Cancer incidence death statistic sources: American Cancer Society and the National Cancer Institute. All figures rounded to nearest thousand.

The present inventors recognize that SRC-3 is overexpressed in an estimated 322,000 new cancer cases and 91,000 cancer deaths in the US each year (Table 1) and that experimental targeting of SRC-3 limits breast cancer cell growth and restores the ability of SERMs to block cancer cells growth; that siRNA-mediated disruption of SRC-3 expression in BT-474 breast cancer cells restores the growth inhibitory effects of 4-hydroxytamoxifen (12); that siRNA-mediated disruption of SRC-3 expression also impairs epidermal growth factor (EGF) activity in a variety of cell lines (13); and that siRNA targeting of SRC-3 leads to reduced transcriptional activity of E2F, impairing the expression of genes important for entry into S phase (14).

The present inventor also recognize that overexpression of SRC-3 promotes prostate cancer cell growth, while in SRC-3 knockout mice, AKT signaling is downregulated (15,16); and that transgenic mice that overexpress SRC-3 develop spontaneous malignant mammary tumors (17); in contrast, SRC-3 knockout mice are resistant to chemical carcinogen-induced and viral-induced mammary tumorigenesis; furthermore, that SRC-3−/− mice are resistant to induced prostate cancer progression (18). The present inventors also appreciate that many advanced hormone refractory breast cancers cease to express ERα and that agents that reduce SRC-3 cellular protein concentration are more inclusive and able to function as anticancer agents in both ERα positive or negative breast cancers.

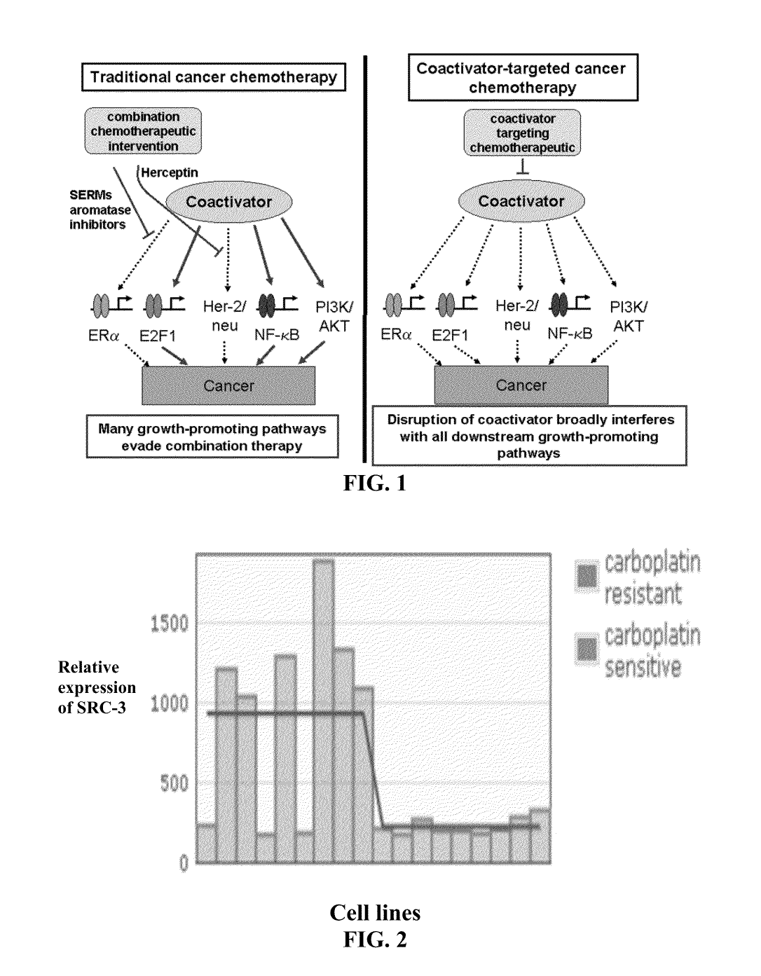

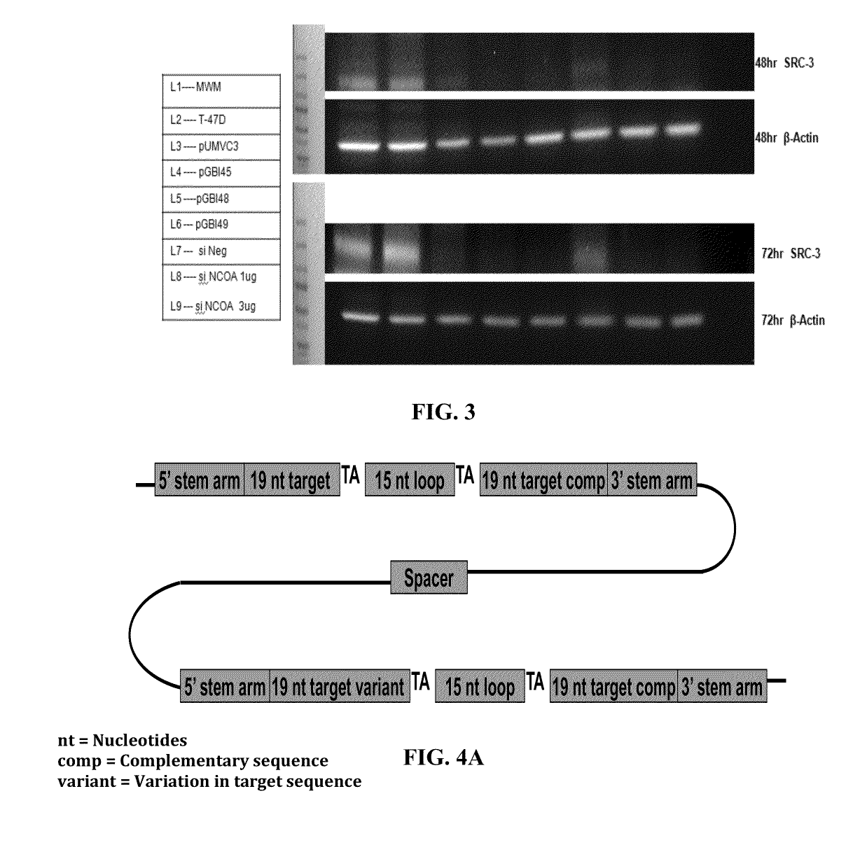

The present inventors recognize that most cancers are highly adaptable and are frequently able to evade the growth-inhibiting action of individual anti-cancer agents; that growth-promoting pathways such as HER2/neu, PI3/AKT, NF-κB frequently become up-regulated in breast cancers in response to treatment with anti-estrogens; and that with so many growth-promoting mechanisms available to it, cancer cells can evade single chemotherapeutic agents that target discrete growth factor pathways. The inventor recognize in particular that because SRC-3 is a central steroid hormone and growth factor signaling integrator as noted above, the response of cancer cells to agents that target SRC-3 is different, because SRC-3 receives growth signaling information by kinases in the PI3/AKT16, NFkB19, PKCi, PKCz20 and other growth factor signaling systems. Phosphorylation of SRC-3 by these kinases then licenses SRC-3 to function as a coactivator for transcription factors such as ERα, NF-kB and E2F114. The present inventors recognize that because of SRC-3's central position at the hub of multiple growth factor signaling pathways, a bishSRC-3 simultaneously interferes with the activity of alternative growth signaling pathways that might lead to cancer chemotherapy resistance (FIG. 1). One way to clearly demonstrate this concept is to recognize that the response of human ovarian cancer patients' response to carboplatin monotherapy is strongly correlated with SRC-3 expression levels; and that microarray analysis retrieved from the European Bioinformatics Institute web-based database reveals that ovarian tumor resistance to treatment with the genotoxic agent carboplatin is strongly correlated with resistance to carboplatin (FIG. 2).

Regarding bifunctional small hairpin RNA interference vectors to target SRC-3 protein expression, the present inventors recognize that RNA interference is a natural cellular regulatory process that inhibits gene expression by transcriptional, post-transcriptional, and translational mechanisms; and that synthetic approaches that emulate this process (small interfering RNA, short hairpin RNA) have been shown to be similarly effective in this regard.

SEQ ID NO: 1 represents the human nuclear receptor coactivator 3 (NCOA3, aka SRC-3), transcript variant 1, mRNA.

(SEQ ID NO: 1)
ATATCCCAGTGGCCCCCGTGCGGCGACTTTAGCTGCTGCTGTCTCAGCCGCTCCA
CAGCGACGGCGGCGGCTGCGGCTTAGTCGGTGGCGGCCGGCGGCGGCTGCGGGCTGAG
CGGCGAGTTTCCGATTTAAAGCTGAGCTGCGAGGAAAATGGCGGCGGGAGGATCAAAA
TACTTGCTGGATGGTGGACTCAGAGACCAATAAAAATAAACTGCTTGAACATCCTTTGA
CTGGTTAGCCAGTTGCTGATGTATATTCAAGATGAGTGGATTAGGAGAAAACTTGGATCCA
CTGGCCAGTGATTCACGAAAACGCAAATTGCCATGTGATACTCCAGGACAAGGTCTTACCTGC
AGTGGTGAAAAACGGAGACGGGAGCAGGAAAGTAAATATATTGAAGAATTGGCTGAGCTGAT
ATCTGCCAATCTTAGTGATATTGACAATTTCAATGTCAAACCAGATAAATGTGCGATTTTAAAG
GAAACAGTAAGACAGATACGTCAAATAAAAGAGCAAGGAAAAACTATTTCCAATGATGATGAT
GTTCAAAAAGCCGATGTATCTTCTACAGGGCAGGGAGTTATTGATAAAGACTCCTTAGGACCG
CTTTTACTTCAGGCATTGGATGGTTTCCTATTTGTGGTGA [symbol] [symbol] [symbol] [symbol]
CAGAAAATGTCACACAATACCTGCAATATAAGCAAGAGGACCTGGTTAACACAAGTGTT
TACAATATCTTACATGAAGAAGACAGAAAGGATTTTCTTAAGAATTTACCAAAATCTACAGTTAA
TGGAGTTTCCTGGACAAATGAGACCCAAAGACAAAAAAGCCATACATTTAATTGCCGTATGTT
GATGAAAACACCACATGATATTCTGGAAGACATAAACGCCAGTCCTGAAATGCGCCAGAGATA
TGAAACAATGCAGTGCTTTGCCCTGTCTCAGCCACGAGCTATGATGGAGGAAGGGGAAGATT
TGCAATCTTGTATGATCTGTGTGGCACGCCGCATTACTACAGGAGAAAGAACATTTCCATCAA
ACCCTGAGAGCTTTATTACCAGACATGATCTTTCAGGAAAGGTTGTCAATATAGATACAAATTC
ACTGAGATCCTCCATGAGGCCTGGCTTTGAAGATATAATCCGAAGGTGTATTCAGAGATTTTTT
AGTCTAAATGATGGGCAGTCATGGTCCCAGAAACGTCACTATCAAGAAGCTTATCTTAATGGC
CATGCAGAAACCCCAGTATATCGATTCTCGTTGGCTGATGGAACTATAGTGACTGCACAGACA
A [symbol] [symbol] [symbol] TCCTGTAACAAATGATCGACATGGCTTTGTCTCAACCCACTTC
CTTCAGAGAGAACAGAATGGATATAGACCAAACCCAAATCCTGTTGGACAAGGGATTAGACCA
CCTATGGCTGGATGCAACAGTTCGGTAGGCGGCATGAGTATGTCGCCAAACCAAGGCTTACA
GATGCCGAGCAGCAGGGCCTATGGCTTGGCAGACCCTAGCACCACAGGGCAGATGAGTGGA
GCTAGGTATGGGGGTTCCAGTAACATAGCTTCATTGACCCCTGGGCCAGGCATGCAATCACC
ATCTTCCTACCAGAACAACAACTATGGGCTCAACATGAGTAGCCCCCCACATGGGAGTCCTG
GTCTTGCCCCAAACCAGCAGAATATCATGATTTCTCCTCGTAATCGTGGGAGTCCAAAGATAG
CCTCACATCAGTTTTCTCCTGTTGCAGGTGTGCACTCTCCCATGGCATCTTCTGGCAATACTG
GGAACCACAGCTTTTCCAGCAGCTCTCTCAGTGCCCTGCAAGCCATCAGTGAAGGTGTGGGG
ACTTCCCTTTTATCTACTCTGTCATCACCAGGCCCCAAATTGGATAACTCTCCCAATATGAATA
TTACCCAACCAAGTAAAGTAAGCAATCAGGATTCCAAGAGTCCTCTGGGCTTTTATTGCGACC
AAAATCCAGTGGAGAGTTCAATGTGTCAGTCAAATAGCAGAGATCACCTCAGTGACAAAGAAA
GTAAGGAGAGCAGTGTTGAGGGGGCAGAGAATCAAAGGGGTCCTTTGGAAAGCAAAGGTCA
TAAAAAATTACTGCAGTTACTTACCTGTTCTTCTGATGACCGGGGTCATTCCTCCTTGACCAAC
TCCCCCCTAGATTCAAGTTGTAAAGAATCTTCTGTTAGTGTCACCAGCCCCTCTGGAGTCTCC
TCCTCTACATCTGGAGGAGTATCCTCTACATCCAATATGCATGGGTCACTGTTACAAGAGAAG
CACCGGATTTTGCACAAGTTGCTGCAGAATGGGAATTCACCAGCTGAGGTAGCCAAGATTACT
GCAGAAGCCACTGGGAAAGACACCAGCAGTATAACTTCTTGTGGGGACGGAAATGTTGTCAA
GCAGGAGCAGCTAAGTCCTAAGAAGAAGGA [symbol] [symbol] [symbol] TACCTGCTGGAC
AGGGATGATCCTAGTGATGCACTCTCTAAAGAACTACAGCCCCAAGTGGAAGGAGTGGATAA
TAAAATGAGTCAGTGCACCAGCTCCACCATTCCTAGCTCAAGTCAAGAGAAAGACCCTAAAAT
TAAGACAGAGACAAGTGAAGAGGGATCTGGAGACTTGGATAATCTAGATGCTATTCTTGGTGA
TCTGACTAGTTCTGACTTTTACAATAATTCCATATCCTCAAATGGTAGTCATCTGGGGACTAAG
CAACAGGTGTTTCAAGGAACTAATTCTCTGGGTTTGAAAAGTTCACAGTCTGTGCAGTCTATTC
GTCCTCCATATAACCGAGCAGTGTCTCTGGATAGCCCTGTTTCTGTTGGCTCAAGTCCTCCAG
TAAAAAATATCAGTGCTTTCCCCATGTTACCAAAGCAACCCATGTTGGGTGGGAATCCAAGAA
TGATGGATAGTCAGGAAAATTATGGCTCAAGTATGGGTGGGCCAAACCGAAATGTGACTGTG
ACTCAGACTCCTTCCTCAGGAGACTGGGGCTTACCAAACTCAAAGGCCGGCAGAATGGAACC
TATGAATTCAAACTCCATGGGAAGACCAGGAGGAGATTATAATACTTCTTTACCCAGACCTGC
ACTGGGTGGCTCTATTCCCACATTGCCTCTTCGGTCTAATAGCATACCAGGTGCGAGACCAGT
ATTGCAACAGCAGCAGCAGATGCTTCAAATGAGGCCTGGTGAAATCCCCATGGGAATGGGGG
CTAATCCCTATGGCCAAGCAGCAGCATCTAACCAACTGGGTTCCTGGCCCGATGGCATGTTG
TCCATGGAACAAGTTTCTCATGGCACTCAAAATAGGCCTCTTCTTAGGAATTCCCTGGATGAT
CTTGTTGGGCCACCTTCCAACCTGGAAGGCCAGAGTGACGAAAGAGCATTATTGGACCAGCT
GCACACTCTTCTCAGCAACACAGATGCCACAGGCCTGGAAGAAATTGACAGAGCTTTGGGCA
TTCCTGAACTTGTCAATCAGGGACAGGCATTAGAGCCCAAACAGGATGCTTTCCAAGGCCAA
GAAGCAGCAGTAATGATGGATCAGAAGGCAGGATTATATGGACAGACATACCCAGCACAGGG
GCCTCCAATGCAAGGAGGCTTTCATCTTCAGGGACAATCACCATCTTTTAACTCTATGATGAAT
CAGATGAACCAGCAAGGCAATTTTCCTCTCCAAGGAATGCACCCACGAGCCAACATCATGAG
ACCCCGGACAAACACCCCCAAGCAACTTAGAATGCAGCTTCAGCAGAGGCTGCAGGGCCAG
CAGTTTTTGAATCAGAGCC [symbol] [symbol] [symbol] AATGGAAAACCCTACTGCTGGTGG
TGCTGCGGTGATGAGGCCTATGATGCAGCCCCAGGTGAGCTCCCAGCAGGGTTTTCTTAATG
CTCAAATGGTCGCCCAACGCAGCAGAGAGCTGCTAAGTCATCACTTCCGACAACAGAGGGTG
GCTATGATGATGCAGCAGCAGCAGCAGCAGCAACAGCAGCAGCAGCAGCAGCAGCAGCAGC
AACAGCAACAGCAACAGCAACAGCAGCAACAGCAGCAAACCCAGGCCTTCAGCCCACCTCCT
AATGTGACTGCTTCCCCCAGCATGGATGGGCTTTTGGCAGGACCCACAATGCCACAAGCTCC
TCCGCAACAGTTTCCATATCAACCAAATTATGGAATGGGACAACAACCAGATCCAGCCTTTGG
TCGAGTGTCTAGTCCTCCCAAT [symbol] [symbol] [symbol] TGGGTCCCTCCCAGAATCCCA
TGATGCAACACCCGCAGGCTGCATCCATCTATCAGTCCTCAGAAATGAAGGGCTGGCCATCA
GGAAATTTGGCCAGGAACAGCTCCTTTTCCCAGCAGCAGTTTGCCCACCAGGGGAATCCTGC
AGTGTATAGTATGGTGCACATGAATGGCAGCAGTGGTCACATGGGACAGATGAACATGAACC
CCATGCCCATGTCTGGCATGCCTATGGGTCCTGATCAGAAATACTGCTGACATCTCTGCACC
AGGACCTCTTAAGGAAACCACTGTACAAATGACACTGCACTAGGATTATTGGGAAGGA
ATCATTGTTCCAGGCATCCATCTTGGAAGAAAGGACCAGCTTTGAGCTCCATCAAGGGT
ATTTTAAGTGATGTCATTTGAGCAGGACTGGATTTTAAGCCGAAGGGCAATATCTACGT
GTTTTTCCCCCCTCCTTCTGCTGTGTATCATGGTGTTCAAAACAGAAATGTTTTTTGGCA
TTCCACCTCCTAGGGATATAATTCTGGAGACATGGAGTGTTACTGATCATAAAACTTTT
GTGTCACTTTTTTCTGCCTTGCTAGCCAAAATCTCTTAAATACACGTAGGTGGGCCAGA
GAACATTGGAAGAATCAAGAGAGATTAGAATATCTGGTTTCTCTAGTTGCAGTATTGG
ACAAAGAGCATAGTCCCAGCCTTCAGGTGTAGTAGTTCTGTGTTGACCCTTTGTCCAGT
GGAATTGGTGATTCTGAATTGTCCTTTACTAATGGTGTTGAGTTGCTCTGTCCCTATTAT
TTGCCCTAGGCTTTCTCCTAATGAAGGTTTTCATTTGCCATTCATGTCCTGTAATACTTC
ACCTCCAGGAACTGTCATGGATGTCCAAATGGCTTTGCAGAAAGGAAATGAGATGACA
GTATTTAATCGCAGCAGTAGCAAACTTTTCACATGCTAATGTGCAGCTGAGTGCACTTT
ATTTAAAAAGAATGGATAAATGCAATATTCTTGAGGTCTTGAGGGAATAGTGAAACAC
ATTCCTGGTTTTTGCCTACACTTACGTGTTAGACAAGAACTATGATTTTTTTTTTTAAAG
TACTGGTGTCACCCTTTGCCTATATGGTAGAGCAATAATGCTTTTTAAAAATAAACTTC
TGAAAACCCAAGGCCAGGTACTGCATTCTGAATCAGAATCTCGCAGTGTTTCTGTGAAT
AGATTTTTTTGTAAATATGACCTTTAAGATATTGTATTATGTAAAATATGTATATACCTT
TTTTTGTAGGTCACAACAACTCATTTTTACAGAGTTTGTGAAGCTAAATATTTAACATTG
TTGATTTCAGTAAGCTGTGTGGTGAGGCTACCAGTGGAAGAGACATCCCTTGACTTTTG
TGGCCTGGGGGAGGGGTAGTGCTCCACAGCTTTTCCTTCCCCACCCCCCAGCCTTAGAT
GCCTCGCTCTTTTCAATCTCTTAATCTAAATGCTTTTTAAAGAGATTATTTGTTTAGATG
TAGGCATTTTAATTTTTTAAAAATTCCTCTACCAGAACTAAGCACTTTGTTAATTTGGGG
GGAAAGAATAGATATGGGGAAATAAACTTAAAAAAAAATCAGGAATTTAAAAAAACG
AGCAATTTGAAGAGAATCTTTTGGATTTTAAGCAGTCCGAAATAATAGCAATTCATGGG
CTGTGTGTGTGTGTGTATGTGTGTGTGTGTGTGTGTATGTTTAATTATGTTACCTTTTCA
TCCCCTTTAGGAGCGTTTTCAGATTTTGGTTGCTAAGACCTGAATCCCATATTGAGATCT
CGAGTAGAATCCTTGGTGTGGTTTCTGGTGTCTGCTCAGCTGTCCCCTCATTCTACTAAT
GTGATGCTTTCATTATGTCCCTGTGGATTAGAATAGTGTCAGTTATTTCTTAAGTAACTC
AGTACCCAGAACAGCCAGTTTTACTGTGATTCAGAGCCACAGTCTAACTGAGCACCTTT
TAAACCCCTCCCTCTTCTGCCCCCTACCACTTTTCTGCTGTTGCCTCTCTTTGACACCTGT
TTTAGTCAGTTGGGAGGAAGGGAAAAATCAAGTTTAATTCCCTTTATCTGGGTTAATTC
ATTTGGTTCAAATAGTTGACGGAATTGGGTTTCTGAATGTCTGTGAATTTCAGAGGTCT
CTGCTAGCCTTGGTATCATTTTCTAGCAATAACTGAGAGCCAGTTAATTTTAAGAATTT
CACACATTTAGCCAATCTTTCTAGATGTCTCTGAAGGTAAGATCATTTAATATCTTTGAT
ATGCTTACGAGTAAGTGAATCCTGATTATTTCCAGACCCACCACCAGAGTGGATCTTAT
TTTCAAAGCAGTATAGACAATTATGAGTTTGCCCTCTTTCCCCTACCAAGTTCAAAATA
TATCTAAGAAAGATTGTAAATCCGAAAACTTCCATTGTAGTGGCCTGTGCTTTTCAGAT
AGTATACTCTCCTGTTTGGAGACAGAGGAAGAACCAGGTCAGTCTGTCTCTTTTTCAGC
TCAATTGTATCTGACCCTTCTTTAAGTTATGTGTGTGGGGAGAAATAGAATGGTGCTCT
TATCTTTCTTGACTTTAAAAAAATTATTAAAAACAAAAAAAAAATAAATTTTTTTGCAA
TCCTTTCCTCAGACCTGGCTCCAGGCTAACTGGAAGGCAGCACTCCCTTTTTTATATAGT
AGAAAAATGAAGTTTATTATAAGTTTTTATATTTTCTACTTGTTCATTTGGTGCAAACTC
AAGATTTCTTTTAATAGGTGCAGTCTTTGAGATAATTTGTTTTTACCTGTATTGCCCTTT
ATCTTTTTTAGGTAATTCTTTGTACTCCTGCTGTCTACCTCTCCTCACACCCCAGCACCC
CCCATTTTTTCAAACCTTGGTATCTGTTGGGTGAACAGTATAATCTTTTCATCTGCTTTT
AGAATGTGGGATATTTCCAGTACCTACTTTTTTTTTTTTTTTTTGCTGAATCCAAAGATA
TATAAATAAAATATATATATTTTATAAAGATCAGAATGATATAAAGGAGATACATGTTT
CTTCCTTTAAAAAATAAACGGAAGTTACATTGTTAATGTTCATATTATGATGCCACTTTT
CTAAACTGCATCTGGATTGAAAGGTGTAAATATCAATAACAGTGCTACTTAGTTATCAG
TATTTAATATCTGAGGTGAGTTGGGGGTATCTATATTAGGGGTAGGGTATTACAGAAGA
TAATTGGCTTGATGTCCTAGAAGTTCTTTGATCCAGAGGTGGGTGCAGCTGAAAGTAAA
CAGAATGGATTGCCAGTTACATGTATGCCTGCCCAGTTCCCTTTTTATTTGCAGAAGCT
GTGAGTTTTGTTCACAATTAGGTTCCTAGGAGCAAAACCTCAAGGATTGATTTATTGTT
TTCAACTCCAAGGCACACTGTTAATAAACGAGCAGGGTGTTTTCTCTCTTCCTTTCTAAT
ATATGGAGTTTCGAAGAATAAAATATGAGAGCAATATTTAAATTCTCAGGAATTGACTT
ATACTCTTGAGAATGAATTCAGTTTCAATCAAGTTTACATTATGTTGCTTAAAAAAATA
GAAATTATTCTTTATCTTGCAAAGAATTGAAACCACATGAAATGACTTATGGGGGATGG
TGAGCTGTGACTGCTTTGCTGACCATTTTGGATGTCATTGTAAATAAAGGTTTCTATTTA
AAATTGGA.

The five double-underlined regions in SEQ ID NO: 1 represent the target sites and the italicized region is the coding region.

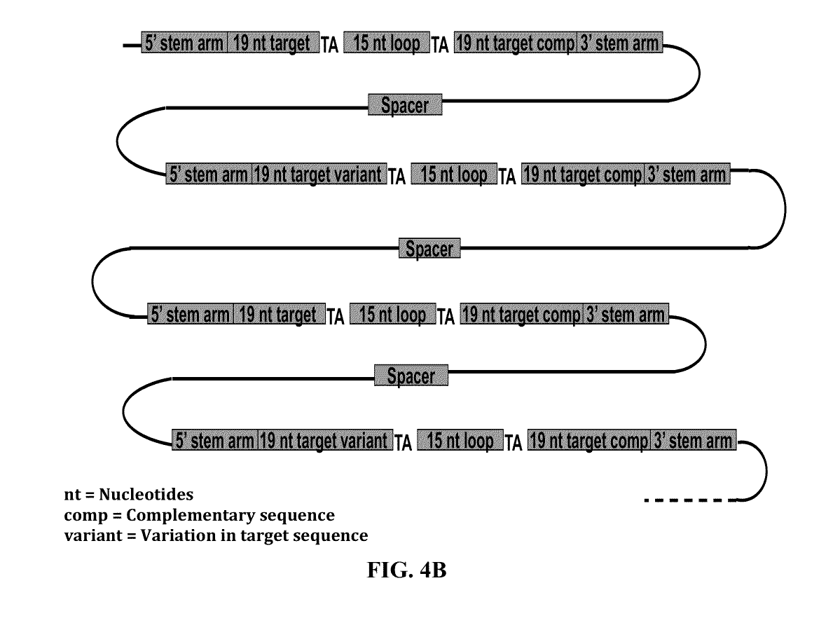

Bifunctional shRNAs targeting SRC-3 are highly effective and have the advantage of causing RNAi at concentrations significantly lower than conventional shRNA or siRNA. The present inventors have developed bishRNAs to optimally target SRC-3 to reduce its expression. Three separate bishSRC-3 vectors targeting different regions of the SRC-3 mRNA are effective in reducing SRC-3 protein levels below the level of detection by Western analysis (FIG. 3).

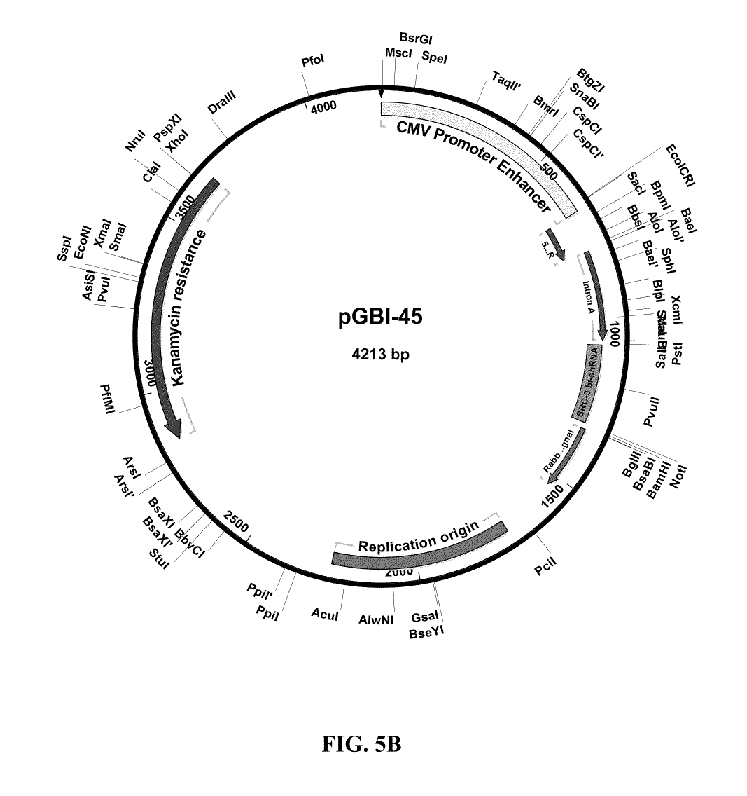

The sequences of the bi-shRNA-NCOA3 of the present invention are shown herein below and the corresponding circular maps are presented in FIGS. 5A-5F:

pGBI-44 (SEQ ID NO: 2):

TCGACTGCTGTTGAAGTGAGCGCCGTTGTCAATATAGATACAATAGTGAAGCCA CAGATGTATTGTATCTATATTGACAACGTTGCCTACTGCCTCGGAAGCAGCTCACTACA TTACTCAGCTGTTGAAGTGAGCGCCGTTGTCAATGCTGATCCAATAGTGAAGCCACAGA TGTATTGTATCTATATTGACAACGTTGCCTACTGCCTCGGAAGCTTAATAAAGGATCTT TTATTTTCATTGGC. The double underlined sequence region corresponds to the sense sequence (nucleotide 1090-1108), wherein the underlined region corresponds to the antisense sequence.

pGBI-45 (SEQ ID NO: 3):

TCGACTGCTGTTGAAGTGAGCGCCAAAGCAAACTCTTCCGAAATAGTGAAGCCA CAGATGTATTTCGGAAGAGTTTGCTTTGTTGCCTACTGCCTCGGAAGCAGCTCACTACA TTACTCAGCTGTTGAAGTGAGCGCCAAAGCAAATACTTCTGAAATAGTGAAGCCACAG ATGTATTTCGGAAGAGTTTGCTTTGTTGCCTACTGCCTCGGAAGCTTAATAAAGGATCT TTTATTTTCATTGGC. The double underlined sequence region corresponds to the sense sequence (nucleotide 1304-1322), wherein the underlined region corresponds to the antisense sequence.

pGBI-46 (SEQ ID NO: 4):

TCGACTGCTGTTGAAGTGAGCGCCGTTGTCAATATAGATACAATAGTGAAGCCA CAGATGTATTGTATCTATATTGACAACGTTGCCTACTGCCTCGGAAGCAGCTCACTACA TTACTCAGCTGTTGAAGTGAGCGCCGAGGAAGAGAGAGGTAAGTTAGTGAAGCCACAG ATGTAACTGACCTGGTTCTTCCTCGTTGCCTACTGCCTCGGAAGCTTAATAAAGGATCT TTTATTTTCATTGGC. The double underlined sequence region corresponds to the sense sequence, wherein the underlined region corresponds to the antisense sequence.

pGBI-47 (SEQ ID NO: 5):

TCGACTGCTGTTGAAGTGAGCGCCAAAGCAAACTCTTCCGAAATAGTGAAGCCA CAGATGTATTTCGGAAGAGTTTGCTTTGTTGCCTACTGCCTCGGAAGCAGCTCACTACA TTACTCAGCTGTTGAAGTGAGCGCCGAGGAAGAGAGAGGTAAGTTAGTGAAGCCACAG ATGTAACTGACCTGGTTCTTCCTCGTTGCCTACTGCCTCGGAAGCTTAATAAAGGATCT TTTATTTTCATTGGC. The double underlined sequence region corresponds to the sense sequence, wherein the underlined region corresponds to the antisense sequence.

pGBI-48 (SEQ ID NO: 6):

TCGACTGCTGTTGAAGTGAGCGCCCCTATATGGTAGAGCAATATAGTGAAGCCA CAGATGTATATTGCTCTACCATATAGGGTTGCCTACTGCCTCGGAAGCAGCTCACTACA TTACTCAGCTGTTGAAGTGAGCGCCCCTATATGCATGATCAATATAGTGAAGCCACAGA TGTATATTGCTCTACCATATAGGGTTGCCTACTGCCTCGGAAGCTTAATAAAGGATCTT TTATTTTCATTGGC. The double underlined sequence region corresponds to the sense sequence, wherein the underlined region corresponds to the antisense sequence.

pGBI-49 (SEQ ID NO: 7):

TCGACTGCTGTTGAAGTGAGCGCCGGAAATGAGATGACAGTATTAGTGAAGCCA CAGATGTAATACTGTCATCTCATTTCCGTTGCCTACTGCCTCGGAAGCAGCTCACTACA TTACTCAGCTGTTGAAGTGAGCGCCGGAAATGACTAGACACTATTAGTGAAGCCACAG ATGTAATACTGTCATCTCATTTCCGTTGCCTACTGCCTCGGAAGCTTAATAAAGGATCTTT TATTTTCATTGGC. The double underlined sequence region corresponds to the sense sequence (nucleotide 1090-1108), wherein the underlined region corresponds to the antisense sequence.

pGBI-40 (SEQ ID NO: 8):

TCGACTGCTGTTGAAGTGAGCGCCATGGAAGGTACAGGAATATTAGTGAAGCCA CAGATGTAATATTCCTGTACCTTCCATGTTGCCTACTGCCTCGGAAGCAGCTCACTACA TTACTCAGCTGTTGAAGTGAGCGCCATGGAAGGACTAGTAATATTAGTGAAGCCACAG ATGTAATATTCCTGTACCTTCCATGTTGCCTACTGCCTCGGAAGCTTAATAAAGGATCTT TTATTTTCATTGGC. The double underlined sequence region corresponds to the sense sequence (nucleotide 1684-1702), wherein the underlined region corresponds to the antisense sequence.

pGBI-41 (SEQ ID NO: 9):

TCGACTGCTGTTGAAGTGAGCGCCTCATGGGAATTCATATCATTAGTGAAGCCA CAGATGTAATGATATGAATTCCCATGAGTTGCCTACTGCCTCGGAAGCAGCTCACTACA TTACTCAGCTGTTGAAGTGAGCGCCTCATGGGACAACATCTCATTAGTGAAGCCACAG ATGTAATGATATGAATTCCCATGAGTTGCCTACTGCCTCGGAAGCTTAATAAAGGATCT TTTATTTTCATTGGC. The double underlined sequence region corresponds to the sense sequence (nucleotide 1331-1349), wherein the underlined region corresponds to the antisense sequence.

pGBI-42 (SEQ ID NO: 10):

TCGACTGCTGTTGAAGTGAGCGCCCCACCAATCAGAAACAGTATAGTGAAGCCA CAGATGTATACTGTTTCTGATTGGTGGGTTGCCTACTGCCTCGGAAGCAGCTCACTACA TTACTCAGCTGTTGAAGTGAGCGCCCCACCAATGTCAAATAGTATAGTGAAGCCACAG ATGTATACTGTTTCTGATTGGTGGGTTGCCTACTGCCTCGGAAGCTTAATAAAGGATCT TTTATTTTCATTGGC. The double underlined sequence region corresponds to the sense sequence (nucleotide 2791-2809), wherein the underlined region corresponds to the antisense sequence.

pGBI-43 (SEQ ID NO: 11):

TCGACTGCTGTTGAAGTGAGCGCCGGAGGAGATTGATAGAGCCTAGTGAAGCCA CAGATGTAGGCTCTATCAATCTCCTCCGTTGCCTACTGCCTCGGAAGCAGCTCACTACA TTACTCAGCTGTTGAAGTGAGCGCCGGAGGAGACATATACAGCCTAGTGAAGCCACAG ATGTAGGCTCTATCAATCTCCTCCGTTGCCTACTGCCTCGGAAGCTTAATAAAGGATCT TTTATTTTCATTGGC. The double underlined sequence region corresponds to the sense sequence (nucleotide 3361-3381), wherein the underlined region corresponds to the antisense sequence.

In sum, the present inventors recognize that SRC-3 is an essential breast cancer oncogene whose transcriptional activation state and cellular protein concentration are critical parameters in determining the coactivator's biological and oncogenic actions; and that SRC-3 bishRNAs targeting SRC-3 are highly effective chemotherapeutic agents and have a high potential to overcome anti-estrogen and anti-HER2 resistance of breast cancers.

The present inventors recognize that cancers typically respond more favorably when treated simultaneously with two or more anticancer agents with distinct mechanisms of action; that cancers typically achieve uncontrolled growth by activating multiple growth signaling systems while also disabling cell division checkpoints and apoptotic pathways; and that, given the involvement of SRC-3 in many central growth signaling pathways, cancer cells will be less likely to develop resistance in the presence of bishSRC-3s (see FIG. 1).

The inventors also recognize that high levels of SRC-3 in cells leads to a state of “resistance to chemotherapy”; and that bishSRC-3 (also when administered in conjunction with a standard chemotherapeutic agent) will provide a more favorable therapeutic response in patients.

One way to determine how the loss of SRC-3 expression interferes with numerous growth factor pathways in chemotherapy sensitive and resistant breast cancer cells is to assess the effects of bishSRC-3 mediated SRC-3 knockdown on global gene expression in, e.g., 1) tamoxifen sensitive MCF-7 cells (23), 2) tamoxifen resistant BT-474 (12), and HER2 positive, ERα negative SKBr3 cells (24) selected for herceptin-resistance (SKBr3R) by passage in 10 mg/ml herceptin for two weeks by microarray analysis.

The inventor recognize that bioinformatic analysis of this data can be used to assess the pleiotropic impact that loss of SRC-3 function has on different gene expression programs in the cell related to growth, apoptosis and cell cycle control.

The present inventors also recognize that that SRC-3's pleiotropic actions in promoting growth factor signaling are related to chemotherapy resistance and that shRNA-mediated knockdown of SRC-3 makes BT-474 cells sensitive to tamoxifen12.

One way to restore tamoxifen sensitivity in these cells is to evaluate and use one or a combination of the three different bishSRC-3 vectors.

One way to perform a gene expression profiling of untreated and bishSRC-3 treated MCF-7, BT-474 and SKBr3R cells is to determine the effects of loss of SRC-3 expression on gene expression patterns or to test the effect of loss of SRC-3 expression on the tamoxifen sensitive MCF-7 cells in the presence of estradiol, tamoxifen or ethanol vehicle evaluate the effect of bishSRC-3s; in addition, the effects of estradiol and tamoxifen to block BT-474 cell growth in the presence of bishSRC-3 and bishRNA empty vector transfected control vector can be evaluated; in addition, the effect of herceptin (or control treatment) in interfering with the growth of SKBr3R cells can be evaluated in the presence or absence of the bishSRC-3 introduced into these cells.

One method to analyze gene expression is to conduct transcript profile analysis using the Affymetrix GeneChip™ Human Genome U133A 2.0 Array, which represents 14,500 well-characterized human genes. The inventors recognize that this array provides coverage of well-substantiated genes in the transcribed human genome and its more compact size, compared to the previous UG-U133A array, allows for reduced sample volume and increased accuracy. Affymetrix approved reagents and protocols in conjunction with the UG133A array can be utilized to determine transcript-wide profiles for each treatment group (Agilent Technologies, Wilmington, Del.).

One way to determine the pleiotropic impact of SRC-3 bishRNAs on cancer cell growth pathways and its effectiveness in a cell culture-based breast cancer combination chemotherapy paradigm is to electroporate cells with the three established bishSRC-3 vectors (FIG. 3) and treat with tamoxifen or ethanol vehicle (MCF-7 and BT-474) or herceptin (SKBr3R) 72 hours later to look at changes in expression in response to tamoxifen or herceptin treatment in the presence of the bishSRC-3 vectors; eight hours after this, cells can be harvested and RNA extracted for analysis. One way to determine significantly regulated genes is to determine the Log 2 ratio expression differences (upregulated and downregulated) between bishSRC-3 and control cells.

The cell lines discussed above can be electroporated as a batch with either bishSRC-3 or its pUMVC3 control vector and then plated into 96-well plates; and these cells are treated with estradiol, tamoxifen (MCF-7 and BT-474 cells) or herceptin (SKBr3R cells). Two, four and six days later, cell growth is determined using a MTS assay (Promega) according to the manufacturer's instructions. Cell apoptosis is determined using an activated caspase activity assay (ApopTag fluorimetric caspases assay kit, Roche). Mitotracker™ (Invitrogen) staining of the same cells will be used to identify compounds that interfere with mitochondrial function.

One way to determination of off-target effects of bishSRC-3s is to generate dose-response curve data for each of the bishSRC-3 vectors to determine the maximum effective doses and the differences in these dose levels with the ability to interfere with SRC-3 coactivator biology. The present inventors recognize that in order to reduce the odds of targeting other genes, BLAST analyses of the bishSRC-3 sequences can be performed, and that RT-PCR quantitation of potentially targeted genes can be performed to verify that they are not targeted by these vectors. The present inventors also recognize that microarray analyses of the bishSRC-3 treated cell lines reveals patterns of gene expression consistent with the loss of SRC-3 expression that distinct from general shRNA toxicity.

One way to provide valuable information about the effectiveness of bishSRC-3 vectors to restore and/or enhance breast cancer cell sensitivity to tamoxifen (BT-474) or anti-HER2 (SKBR3R) treatment is to employ an in vitro cell culture-based approach, and the findings can also be substantiated in an animal model system; this also demonstrates the effectiveness of these bishSRC-3/chemotherapy combinations.

One way to demonstrate the ability to effectively deliver bishSRC-3 to tumors in living animals, block their growth, and demonstrate the preclinical feasibility involves the use of human breast cancer cell-mouse host xenograft models. These xenograft models also provide an important platform to combine these bishSRC-3 with a bilamellar invaginated vesicle (BIV), and these xenograft models serve to demonstrate that tamoxifen or herceptin and a bishSRC-3+BIV lipoplex can all be effectively combined together to block breast tumors cell growth.

One way to demonstrate that survival and growth is affected by bishSRC-3 upon exposure to appropriate chemotherapeutics (5 mg, 60 day release tamoxifen paraffin pellets in MCF-7 and BT-474 cells) and 16 mg/kg herceptin, delivered via intraperitoneal injection (SKBR3R cells) involves subcutaneous transplantation of MCF-7, BT-474 and SKBr3R cells in to nude mice as xenografts.

One way to test the bishSRC-3s for their ability to inhibit or reverse tumor growth, both alone and in combination with these chemotherapies, is to compare growth of xenografts with no treatment control mice, as well as with tamoxifen and anti-HER agents and the empty bishRNA vector (pUMVC3) over a 60 day period.

The present inventor recognize that, in order afford adequate statistical power to detect a significant difference in time to tumor doubling (or halving) in response to treatment, eight mice per treatment group per agent can be employed; in addition, to tumor growth curves, treated tumors can also be evaluated for changes in proliferation (Ki67), apoptosis (cleaved caspase 3) and the expression of ERα, SRC-3 and HER2.

The present inventors recognize that in vivo and clinically applicable gene knockdown can be hindered by a lack of effective systemic delivery; that small, double-stranded oligonucleotides have circulatory half-lives of seconds to minutes even when chemically modified (25,26); and that most delivery vehicles fall short due to colloidal instability, aggregation, high clearance by non-target organs, immunogenicity, poor in vivo transfection efficiencies, and impaired gene expression.

The present inventors have produced BIV delivery vehicle that have overcome these constraints (27) and are highly effective for systemic therapeutic payload delivery to primary and metastatic human cancers including pancreatic cancer xenograft foci (28).

In one embodiment, the cationic BIV delivery vehicle comprises a manually extruded formulation of biodegradable 1,2-dioleoyl-3-trimethylammoniopropane (DOTAP) and cholesterol. In some embodiments, BIV delivery vehicles do not contain polyethylene glycol (PEG). The present inventors recognize that PEGylation reduces the “sponge effect” of first pass organ non-target retention but also induces steric hindrance and inefficient target cellular uptake (despite decoration). In one embodiment, BIV cationic delivery vehicles have an optimized half-life of five hours and are stable in circulation.

In a further embodiment, nucleic acids encapsulated in these flexible delivery vehicles of 200-450 nm can penetrate the capillary fenestra of the tumor microenvironment, other tight intercellular junctions (e.g., the blood retinal barrier), and permeate large tumors countercurrent to the interstitial pressure gradient. BIV delivery vehicles have attained the highest comparative levels of gene expression documented post-IV injection in mice.

One way to determine the maximum effective dosage to block tumor growth it to deliver bishSRC-3 vectors and controls intraperitoneally one week after tumor xenograft insertion into the host animal at different doses.

Bifunctional shRNA (bi-shRNA) vectors to target the SRC-3 and SRC-1 oncogenes.

Steroid receptor coactivator-3/amplified in breast cancer-1 (SRC-3/AIB-1) and steroid receptor coactivator-1 (SRC-1) are key breast cancer oncogenes that are frequently overexpressed or amplified in estrogen receptor and HER2 positive breast cancers. Experimental targeting of either SRC-3 or SRC-1 has been shown to limit breast cancer cell growth and restore the anti-estrogenic actions of tamoxifen. Here, are utilizing RNA interference (RNAi) technology with bifunctional shRNA (bi-shRNA)-based design for singlet SRC-1 or SRC-3 knockdown (with the capability for duplex construction). Bi-shRNA effectors achieve enhanced target knockdown by simultaneously promoting target mRNA cleavage, mRNA degradation (via p-body sequestration) and translational repression resulting in a lower dose requirement.

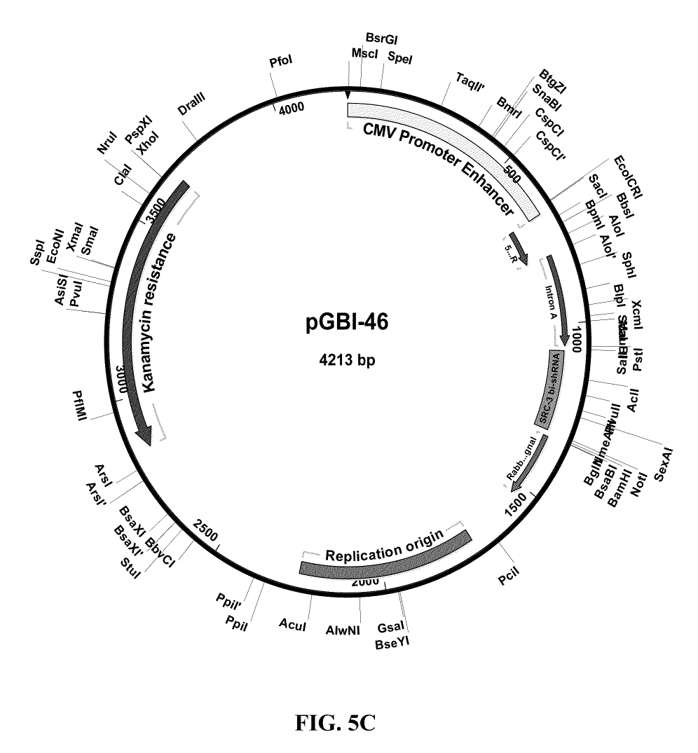

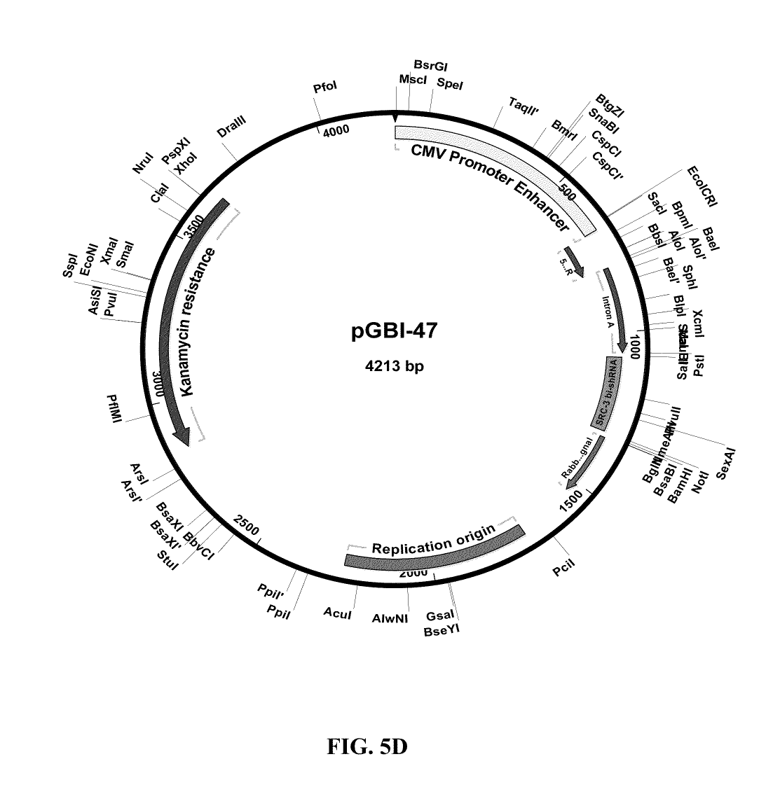

Different SRC-3 and SRC-1 targeting bi-shRNAs were evaluated in cell culture models to identify the most effective construct variants based on their ability to block coactivator expression and breast cancer cell growth. This has led to the identification of SRC-1 and SRC-3 bi-shRNA constructs that are able to effectively reduce the expression of SRC-3 and SRC-1 to low levels in MCF-7 breast cancer cells (via Western immunoblots). First, we examined the effect of SRC-3 bi-shRNA vectors on SRC-3 protein expression in MCF-7 and MDA-MB-231 breast cancer cells (FIGS. 6 and 7). All bi-shRNA vectors were able to reduce SRC-3 protein expression in MCF-7 cells. In MDA-MB-231 cells, bi-shRNA vectors pGBI-45 and pGBI-49 were able to effectively reduce SRC-3 protein expression while bi-shRNA vectors pGBI-46, pGBI-47 and pGBI-48 did so to a lesser extent.

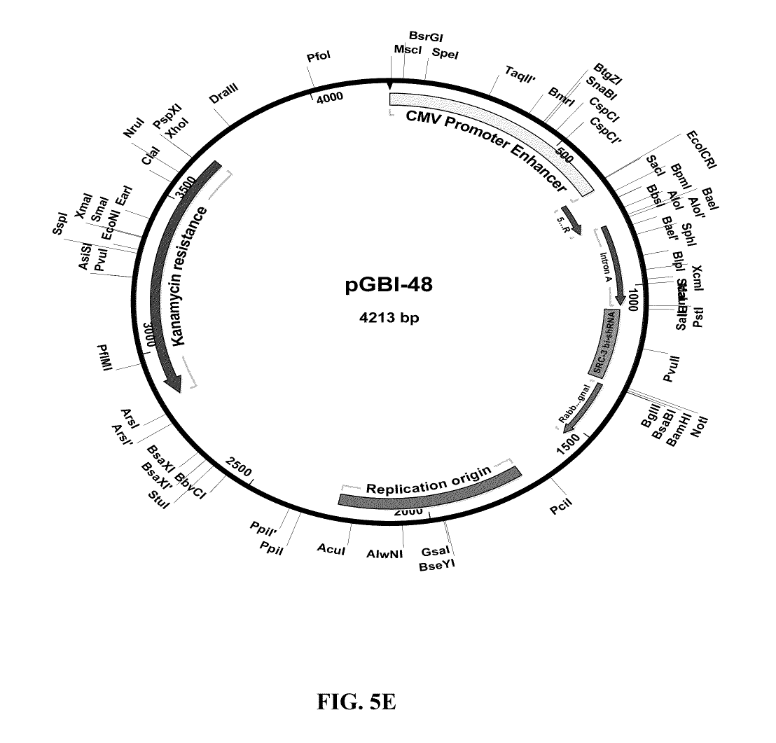

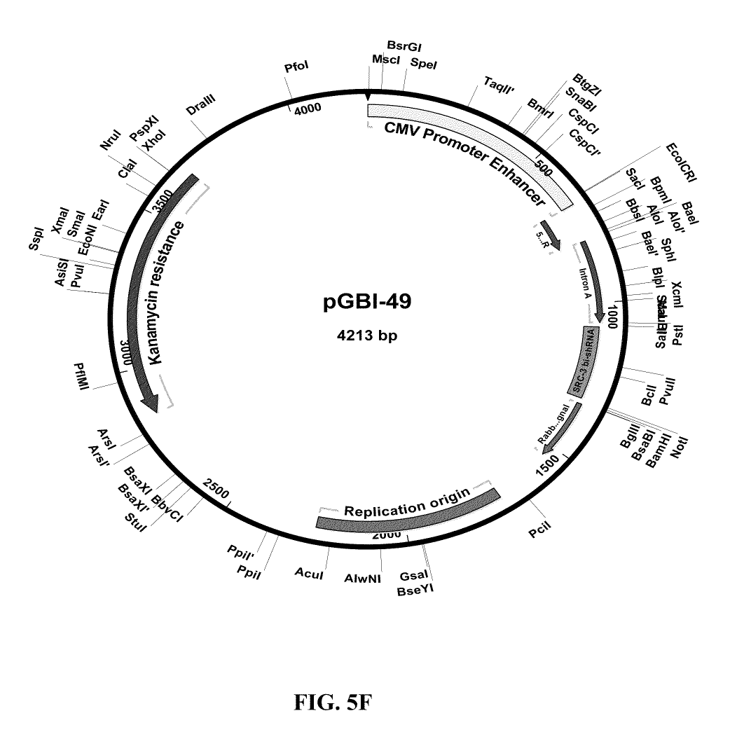

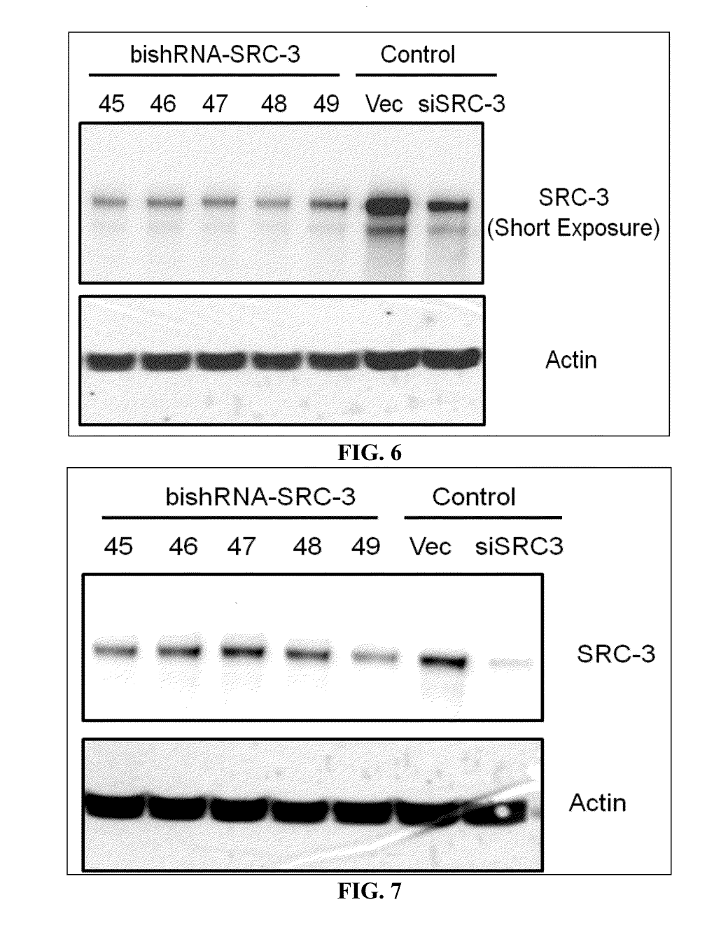

Cell growth assays were performed to examine the ability of SRC-3 and SRC-1 bi-shRNA vectors to block breast cancer cell growth. MCF-7 cells were transfected with SRC-1 and SRC-3 bio-shRNA vectors and their effects on cell proliferation were measured via MTT assay after four (FIG. 8) or five days (FIG. 9). All SRC targeting vectors were able to effectively reduce cell growth in contrast to the negative control (siGFP). Similarly, inhibition of growth is also observed on MDA-MB-231 cells transfected with SRC-1 and SCR-3 bi-shRNA vectors (FIG. 10). These results confirm that bi-shRNA based targeting of SRC-1 and SRC-3 can reduce breast cancer cell growth in vitro.

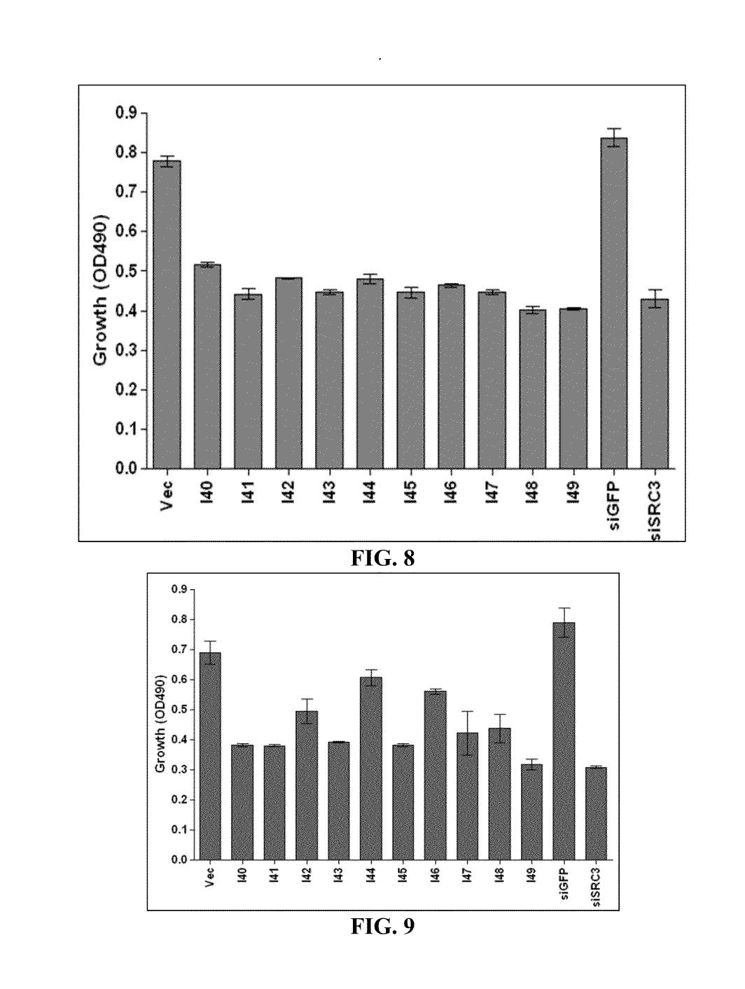

The SRC-3 knockdown was further examined with A-549 lung cancer cell line. SRC-1 and SCR-3 targeting bi-shRNA expression vectors transfected into A-549 cells were able to knockdown SRC-3 protein expression effectively when compared with the empty vector control (FIG. 11). For lung cancer cells, bi-shRNA vectors pGBI-43, pGBI-48 and pGBI-49 are most effective in reducing SRC-3 expression.

In conclusion, SRC-1 and SCR-3 targeting bi-shRNA constructs can effectively reduce the SRC-3 protein expression in both breast cancer cells (MCF-7 and MDA-MB-231 cells) and lung cancer cells (A-549 cells). These constructs can also reduce breast cancer cell growth in vitro. The demonstration on cell growth inhibition for lung cancer cells in vitro is in progress.

The RNA interference bifunctional shRNA (bi-shRNA)-based design constructs for singlet SRC-1 or SRC-3 knockdown (with the capability for duplex construction), or combinations of the same were used to target triple negative breast cancer cells. The Bi-shRNA effectors achieved enhanced target knockdown by simultaneously promoting target mRNA cleavage, mRNA degradation (via p-body sequestration) and translational repression resulting in a lower dose requirement. The inventors evaluated different SRC-3 and SRC-1 targeting bi-shRNAs in cell culture models to identify the most effective construct variants based on their ability to block coactivator expression and breast cancer cell growth. The SRC-1 and SRC-3 bi-shRNA constructs were able to effectively reduce the expression of SRC-3 and SRC-1 to nearly undetectable levels in MCF-7 breast cancer cells (via Western immunoblots). Cell growth assays also confirm that they are effective in reducing breast cancer cell growth in vitro. The constructs were also evaluated in a mouse metastatic breast cancer xenograft model using a MDA-MB-231 subline selected for its ability to metastasize to the lung. SRC-3 bi-shRNA vectors encapsulated within a novel bilamellar invaginated vesicle (BIV) lipoplex delivery system designed for effective systemic delivery of bi-shRNAs to cancer cell are being used in these experiments. Cross-species design of bi-shRNAs allow the evaluation of the formulation in mouse breast cancer models for efficacy as well as for safety. Given the central roles that SRC-3 and SRC-1 have in breast and other cancers and the lack of clinically available agents to target these key oncogenes, SRC-3/SRC-1 bi-shRNAs are a unique class of gene-based agents to treat breast cancer.

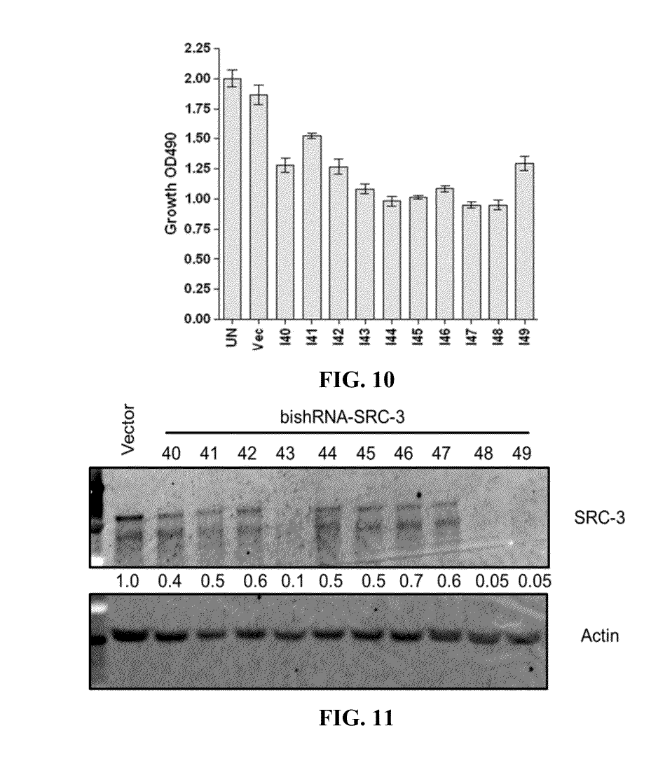

FIG. 12 shows that five different bishSRC-3 vectors can reduce SRC-3 protein expression in MCF-7 and MDA-231 breast cancer cells. bishSRC-3 vectors (pGBI 45-49) or their parent, empty expression vector pUMVC3 (Vec) or a Dharmacon SRC-3 targeting siRNA pool (siSRC-3) were reversed transfected into cells and assayed for SRC-3 protein levels 72 hours later.

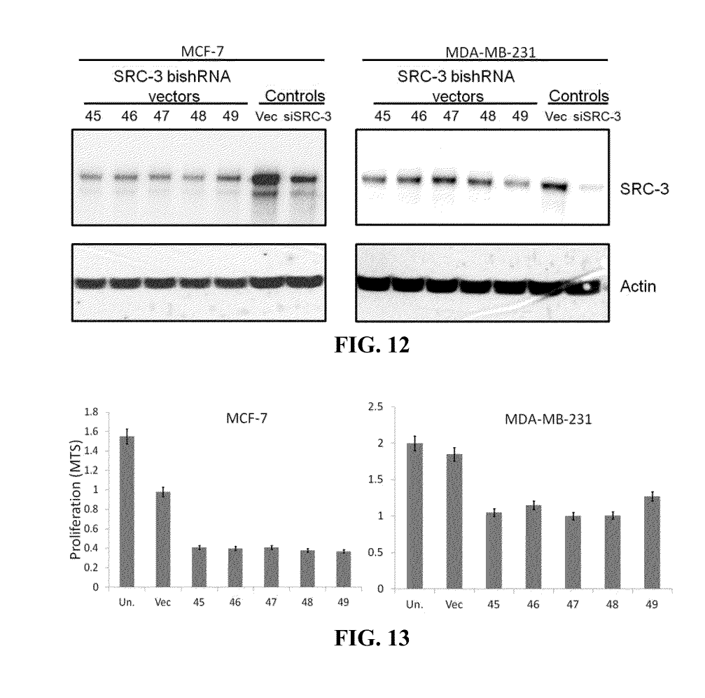

FIG. 13 shows that five different bishSRC-3 vectors can reduce SRC-3 protein expression in MCF-7 and MDA-231 breast cancer cells. Untreated (Un), bishSRC-3 vectors (pGBI 45-49) or their parent, empty expression vector pUMVC3 (Vec) were reversed transfected into cells and assayed for SRC-3 protein levels 72 hours later. Cell proliferation was measured by MTS assay.

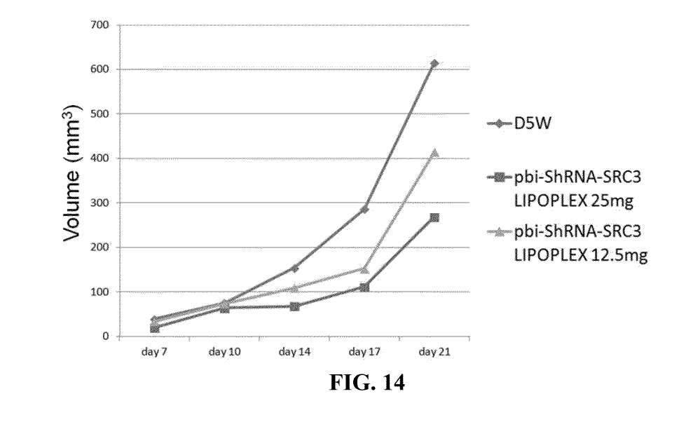

SRC-3 targeting bishRNAs can inhibit breast cancer cell proliferation in a mouse xenograft model system. The SRC-3 bi-shRNA vector was used in a pre-clinical mouse primary tumor growth and metastasis model for its ability to block tumor growth. The SRC-3 targeting bi-shRNA vector (pGBI-45) was compared with a control vector (pUCMV3)(data not shown) or water. This model consists of a subline of the estrogen receptor negative MDA-MB-231 breast cancer cell line that has been selected for its ability to aggressively metastasize to the lung. Twenty-five athymic nude mice were injected with cells at 2×10⁶ per site into the 2nd mammary gland (cleared) with two sites per mice. Mice were then separated into the following groups: (1) D5W (water); (2) 12.5 mg pGBI-45 SRC-3 bishRNA vector; (3) 25 mg pGBI-45 SRC-3 bishRNA vector; (4) 12.5 mg empty expression vector; or (5) 25 mg empty expression vector (data not shown). Results are shown in FIG. 14.

FIG. 14 is a graph that shows that SRC-3 targeting bifunctional shRNA vector pGBI-45 suppresses the primary tumor growth in the mouse LM3 (MDA-MD-231 subline) xenograft model system. Tumor bearing mice were treated once a week with water control (D5W) or SRC-3 bishRNA (pGBI-45) via tail vein injection. Tumor volume was measured on indicated days after initial treatment.

It was found that SRCs are broadly implicated in cancer cell growth. The present inventors developed SRC-based small molecule inhibitors and/or shRNA-based targeting vectors (SMIs) as anti-cancer drugs. Bifunctional short hairpin RNA (bishRNA) plasmid vectors were designed that effectively targeted and down regulated expression of the SRC-3 protein in breast cancer cells. These SRC-3 bishRNA vectors were also able to reduce cell proliferation in both ER+ (MCF-7) and ER− (MDA-MB-231) cell lines in vitro. In conjunction with a bilamellar invaginated vesicle (BIV) lipoplex delivery system, the pGBI-45 SRC-3 targeting bifunctional shRNA vector was able to block breast cancer cell growth in a triple negative breast cancer xenograft model.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 7,282,576: Coactivators in the diagnosis and treatment of breast cancer.
U.S. Patent Publication No. 20070099209: Compositions and Methods for Treating and Diagnosing Cancer.
U.S. Patent Application Publication No 20100286244: RNAi Mediated Knockdown of Numa for Cancer Therapy.
U.S. Patent Application Publication No. 20040086911: Inhibition of Gene Expression in Vertebrates Using Double-Stranded RNA (RNAi).
1. Sunami, E. et al. Estrogen receptor and HER2/neu status affect epigenetic differences of tumor-related genes in primary breast tumors. Breast Cancer Res 10, R46 (2008).
2. Bender, L. M. & Nahta, R. Her2 cross talk and therapeutic resistance in breast cancer. Front Biosci 13, 3906-12 (2008).
3. Culhane, A. C. & Howlin, J. Molecular profiling of breast cancer: transcriptomic studies and beyond. Cell Mol Life Sci 64, 3185-200 (2007).
4. Normanno, N. et al. Target-based therapies in breast cancer: current status and future perspectives. Endocr Relat Cancer 16, 675-702 (2009).
5. Lonard, D. M., Lanz, R. B. & O'Malley, B. W. Nuclear receptor coregulators and human disease. Endocr Rev 28, 575-87 (2007).
6. Lonard, D. M. & O'Malley B, W. Nuclear receptor coregulators: judges, juries, and executioners of cellular regulation. Mol Cell 27, 691-700 (2007).
7. Glaeser, M., Floetotto, T., Hanstein, B., Beckmann, M. W. & Niederacher, D. Gene amplification and expression of the steroid receptor coactivator SRC3 (AIB1) in sporadic breast and endometrial carcinomas. Horm Metab Res 33, 121-6 (2001).
8. Henke, R. T. et al. Overexpression of the nuclear receptor coactivator AIB1 (SRC-3) during progression of pancreatic adenocarcinoma. Clin Cancer Res 10, 6134-42 (2004).
9. Xu, F. P. et al. SRC-3/AIB 1 protein and gene amplification levels in human esophageal squamous cell carcinomas. Cancer Lett 245, 69-74 (2007).
10. Liu, M. Z. et al. Overexpression of AIB1 in nasopharyngeal carcinomas correlates closely with advanced tumor stage. Am J Clin Pathol 129, 728-34 (2008).
11. Schiff, R., Massarweh, S., Shou, J. & Osborne, C. K. Breast cancer endocrine resistance: how growth factor signaling and estrogen receptor coregulators modulate response. Clin Cancer Res 9, 447S-54S (2003).
12. Su, Q. et al. Role of AIB1 for tamoxifen resistance in estrogen receptor-positive breast cancer cells. Oncology 75, 159-68 (2008).
13. Lahusen, T., Fereshteh, M., Oh, A., Wellstein, A. & Riegel, A. T. Epidermal growth factor receptor tyrosine phosphorylation and signaling controlled by a nuclear receptor coactivator, amplified in breast cancer 1. Cancer Res 67, 7256-65 (2007).
14. Louie, M. C., Zou, J. X., Rabinovich, A. & Chen, H. W. ACTR/AIB1 functions as an E2F1 coactivator to promote breast cancer cell proliferation and antiestrogen resistance. Mol Cell Biol 24, 5157-71 (2004).
15. Oh, A. et al. The nuclear receptor coactivator AIB1 mediates insulin-like growth factor I-induced phenotypic changes in human breast cancer cells. Cancer Res 64, 8299-308 (2004).
16. Zhou, H. J. et al. SRC-3 is required for prostate cancer cell proliferation and survival. Cancer Res 65, 7976-83 (2005).
17. Torres-Arzayus, M. I. et al. High tumor incidence and activation of the PI3K/AKT pathway in transgenic mice define AIB1 as an oncogene. Cancer Cell 6, 263-74 (2004).
18. Kuang, S. Q. et al. Mice lacking the amplified in breast cancer 1/steroid receptor coactivator-3 are resistant to chemical carcinogen-induced mammary tumorigenesis. Cancer Res 65, 7993-8002 (2005).
19. Wu, R. C. et al. Regulation of SRC-3 (pCIP/ACTR/AIB-1/RAC-3/TRAM-1) Coactivator activity by I kappa B kinase. Mol Cell Biol 22, 3549-61 (2002).
20. Yi, P. et al. Atypical protein kinase C regulates dual pathways for degradation of the oncogenic coactivator SRC-3/AIB1. Mol Cell 29, 465-76 (2008).
21. Joen, T. Y. et al. Overexpression of stathminl in the diffuse type of gastic cancer and its roles in proliferation and migration of gastric cancer cells. Br J Cancer 102, 710-8.
22. Hsieh, S. Y. et al. Stathminl overexpression associated with polyploidy, tumor-cell invastion, early recurrence, and poor prognosis in human heptatoma. Mol Carcinog 49, 476-87.
23. Lippman, M., Bolan, G. & Huff, K. The effects of estrogens and antiestrogens on hormone-responsive human breast cancer in long-term tissue culture. Cancer Res 36, 4595-601 (1976).
24. Jallal, B., Schlessinger, J. & Ullrich, A. Tyrosine phosphatase inhibition permits analysis of signal transduction complexes in p185HER2/neu-overexpressing human tumor cells. J Biol Chem 267, 4357-63 (1992).
25. Werner, R. G. Gene technology: chances for diagnosis and therapy. Methods Find Exp Clin Pharmacol 16, 525-37 (1994).
26. Crooke, S. T. An overview of progress in antisense therapeutics. Antisense Nucleic Acid Drug Dev 8, 115-22 (1998).
27. Tong, A. W., Zhang, Y. A. & Nemunaitis, J. Small interfering RNA for experimental cancer therapy. Curr Opin Mol Ther 7, 114-24 (2005).
28. Shi, Q. et al. A combinatorial approach for targeted deliver using small molecules and reversible masking to bypass nonspecific uptake in vivo. Gene Ther 17, 1085-97.

What is claimed is:

1. A method for treating triple negative breast cancer comprising administering a therapeutically effective amount of a formulation that includes vector that expresses an SRC-1-specific bifunctional shRNA, an SRC-3-specific bifunctional shRNA, or both, to impair triple negative breast cancer cell growth.

2. The method of claim 1, wherein the formulation further comprises a cationic liposomal preparation.

3. The method of claim 2, wherein the cationic liposomal preparation comprises a single vector that encodes the SRC-1-specific bifunctional shRNA, the SRC-3-specific bifunctional shRNA, or both the SRC-1-specific bifunctional shRNA and the SRC-3-specific bifunctional shRNA.

4. The method of claim 1, wherein the one or more shRNA's is comprises a sequence selected from SEQ ID NO: 2, SEQ ID NO: 3, SEQ. ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or combinations or modifications thereof.

5. The method of claim 1, wherein a sequence arrangement for the shRNA comprises a 5′ stem arm-19 nucleotide target (SRC-3 gene)-TA-15 nucleotide loop-19 nucleotide target complementary sequence-3′ stem arm-Spacer-5′ stem arm-19 nucleotide target variant-TA-15 nucleotide loop-19 nucleotide target complementary sequence-3′ stem arm.

6. The method of claim 1, wherein the one or more polycations is a 10 kDA polyethylene glycol (PEG)-substituted cysteine-lysine 3-mer peptide (CK30PEG10k).

7. The method of claim 1, wherein the compacted DNA nanoparticles are further encapsulated in a liposome.

8. The method of claim 1, wherein the liposome is a bilamellar invaginated vesicle (BIV).

9. The method of claim 1, wherein the triple negative breast cancer is resistant to chemotherapeutic agents.

10. A method of treating a triple negative breast cancer in a human subject comprising the steps of:
identifying a human subject in need for suppression of triple negative breast cancer cell growth; and
administering an expression vector in a therapeutic agent carrier complex to the human subject in an amount sufficient to suppress the growth of a triple negative breast cancer cells;
wherein the expression vector expresses one or more bifunctional short hairpin RNA (shRNA) capable inhibiting an expression of an SRC-1 gene, an SRC-3 gene, or both, wherein the one or more shRNA comprise a bifunctional RNA molecule that activates concurrently both a cleavage-dependent and a cleavage-independent RNA-induced silencing complex for reducing the expression level of the SRC-1 gene, the SRC-3 gene, or both.

11. The method of claim 10, wherein the one or more shRNAs are selected from the group consisting of SEQ ID NO: 2, SEQ ID NO: 3, SEQ. ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and combinations or modifications thereof.

12. The method of claim 10, wherein a sequence arrangement for the shRNA comprises a 5′ stem arm-19 nucleotide target (SRC-3 gene)-TA-15 nucleotide loop-19 nucleotide target complementary sequence-3′ stem arm-Spacer-5′ stem arm-19 nucleotide target variant-TA-15 nucleotide loop-19 nucleotide target complementary sequence-3′ stem arm.

13. The method of claim 10, wherein the therapeutic agent carrier is a compacted DNA nanoparticle or a reversibly masked liposome decorated with one or more receptor targeting moieties, wherein the one or more receptor targeting moieties are small molecule bivalent beta-turn mimics.

14. The method of claim 10, wherein the therapeutic agent carrier is a compacted DNA nanoparticle that is compacted with one or more polycations, wherein the one or more polycations comprises a 10 kDA polyethylene glycol (PEG)-substituted cysteine-lysine 3-mer peptide (CK₃₀PEG10k) or a 30-mer lysine condensing peptide.

15. The method of claim 10, wherein the reversibly masked liposome is a bilamellar invaginated vesicle (BIV).

16. The method of claim 10, wherein the compacted DNA nanoparticles are further encapsulated in a liposome.

17. The method of claim 10, wherein the tumor cell or breast cancer is resistant to tamoxifen therapy.

18. The method of claim 10, further comprising administering tamoxifen.

19. The method of claim 10, further comprising the step of administering the vector before, after, or concurrently as a combination therapy with one or more treatment methods selected from the group consisting of chemotherapy, radiation therapy, surgical intervention, antibody therapy, Vitamin D therapy, or any combinations thereof.

20. The method of claim 10, wherein the triple negative breast cancer is resistant to chemotherapeutic agents.

21. A method of treating one or more cancers resistant to chemotherapy, increasing effectiveness of one or more chemotherapeutic agents, or both in a subject comprising the steps of:
identifying the human or animal subject having the cancer resistant to the chemotherapeutic agents or in need of increased effectiveness of the one or more chemotherapeutic agents, wherein the breast cancer is a triple negative breast cancer; and
administering an expression vector in a therapeutic agent carrier complex to the human or animal subject in an amount sufficient to suppress or inhibit an expression of an SRC-1 gene, an SRC-3 gene, or both in the subject, wherein the expression vector expresses one or more bifunctional short hairpin RNA (shRNA) capable inhibiting the expression of the SRC-1 gene, the SRC-3 gene, or both in one or more triple negative breast cancer cells in the subject via RNA interference, wherein the inhibition results in an enhanced action of the one or more chemotherapeutic agents leading to an apoptosis, an arrested proliferation, or a reduced invasiveness of one or more triple negative breast cancer cells;
wherein the one or more bifunctional shRNA activate a cleavage-dependent and a cleavage-independent RNA-induced silencing complex for reducing the expression level of SRC-1, SRC-3, or both.

22. The method of claim 21, wherein the one or more chemotherapeutic agents comprise platinum drugs, carboplatin, tamoxifen, ER antagonists, or any combinations thereof.

23. The method of claim 21, wherein the cancers are selected from the group consisting of colon, breast, pancreatic, prostate, or any combinations thereof.

24. The method of claim 21, wherein the one or more shRNAs are selected from the group consisting of SEQ ID NO: 2, SEQ ID NO: 3, SEQ. ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and combinations or modifications thereof.

25. The method of claim 21, wherein the vector is administered before, after, or concurrently as with the one or more chemotherapeutic agents.

26. The method of claim 21, wherein the triple negative breast cancer is resistant to chemotherapeutic agents.
